(19) United States
(12) Patent Application Publication
(10) Pub. No.: US 2019/0247051 A1
(43) Pub. Date: Aug. 15, 2019

(54) ACTIVE TEXTILE ENDOGRAFT

(71) Applicant: Fareed Siddiqui, MINNETONKA, MN (US)

(72) Inventor: Fareed Siddiqui, MINNETONKA, MN (US)

(21) Appl. No.: 16/277,152

(22) Filed: Feb. 15, 2019

Related U.S. Application Data
(60) Provisional application No. 62/630,884, filed on Feb. 15, 2018.

(51) Int. Cl. A61B 17/12; A61F 2/07; A61F 2/06

(52) U.S. Cl. CPC A61B 17/12118; A61B 17/12136; A61F 2/07; A61F 2/064; A61F 2002/045; A61F 2002/065; A61F 2002/061; A61F 2002/072; A61F 2210/008; A61F 2210/0014

(57) ABSTRACT

An endograft composed of an active textile material that can intrinsically change shape once placed inside the body after activation of the active textile material. The shape change can allow for the creation of exclusive channels between the endograft and tubular/hollow organs in the body or between other endografts. The shape change can also lead to the creation of the endograft inside the body.

### RELATED APPLICATION

The present application claims the benefit of U.S. Provisional Application No. 62/630,884 which is incorporated herein by reference.

### FIELD OF TECHNOLOGY

Embodiments are directed generally to medical devices, and more particularly to endografts formed of active textiles.

### BACKGROUND OF THE INVENTION

Endografts also referred to as stentgrafts or covered stents are tubular or branched tubular structures usually made of a matrix of metal or bio absorbable materials covered by or attached to a layer of fabric or plastic materials. Endografts are placed inside tubular or hollow structures in the body such as blood vessels, bile ducts, the bronchial tree, the urinary system, gastrointestinal system etc. to maintain patency of these structures and allow for passage of bodily fluids such as blood, succus, bile, respiratory air, urine, stool etc. Endografts also function to exclude the bodily environment outside of the lumen of the endograft. This can be beneficial in the treatment of aneurysmal disease of arteries, for the prevention of tissue ingrowth through the interstices of noncovered stents, for covering tears in the wall of a tubular organ, and for creating bypass channels between two tubular structures through non tubular soft tissues for example in the creation of a transjugular portosystemic shunt through the liver or in the creation of a vascular dialysis graft or a vascular bypass graft.

Current endografts have a predetermined shape and diameter post deployment in the body that is set during their manufacturing process. Therefore, current endografts do not have the ability to intrinsically change shape post deployment. This static nature of current endografts limits their use in the treatment of various diseases. This can be illustrated in the use of current endografts in the treatment of arterial aneurysms.

Endovascular aneurysm repair involves the placement of a fabric covered endograft or modular endograft devices across an aneurysm of an artery usually to exclude the wall of the aneurysm from the pressure of flowing blood to prevent rupture of the aneurysm. When used to treat the abdominal aorta, this procedure is termed an EVAR (endovascular abdominal aortic aneurysm repair) and when used to treat the thoracic aorta the procedure is termed TEVAR (thoracic endovascular aortic/aneurysm repair).

Suitable landing zones proximal and distal to the aneurysm must exist for the safe deployment of an endograft. The endograft fabric must have good wall apposition in these landing zones to prevent leakage of flowing blood around the graft into the aneurysm sac which would continue pressurization on the aneurysm wall and lead to rupture. Suitable landing zones should be devoid of important arterial branches which if covered by the endograft could lead to loss of flow in these arteries and therefore significant organ injury. In the abdomen these branches are the renal, superior mesenteric and celiac arteries. In the thorax, these are the subclavian, carotid, innominate, and coronary arteries.

The greatest limitation to treating all patients with EVAR or TEVAR is the lack of suitable landing zone anatomy. Abdominal aortic aneurysms that involve the origin of the renal arteries (pararenal aneurysms) and mesenteric arteries such as the celiac trunk and superior mesenteric artery (paravisceral aneurysms) cannot be treated by standard EVAR due to a lack of a suitable landing zone. Similarly thoracic aortic aneurysms which involve the aortic arch takeoff vessels and thoracoabdominal aneurysms which involve the mesenteric arteries cannot be treated by standard TEVAR. Lack of suitable landing zone anatomy also limits the use of standard endografts in the ascending thoracic aorta due to risk of injury to the coronary arteries.

Many endovascular strategies have been developed to treat patients with suboptimal landing zone anatomy. Foremost amongst these is the use of fenestrated (FEVAR) and branched endografts. Fenestrated endografts have small holes or fenestrations which can be selected by an angiographic catheter which is then used to select the important branch arteries that require preservation. Upon selection, the artery is then stented with a stentgraft extending from the endograft to the artery. The same is accomplished through preformed graft branches rather than fenestrations in a branched aortic endograft.

There are limitations to current fenestrated endografts. Neck angulation poses a particularly difficult problem, as endograft device orientation and positioning of the fenestration can become extremely difficult. The positioning of the fenestrations has to be precise in order to select the branch artery for which flow needs to be preserved. Significant mismatch between a fenestration and branch artery can lead to complete coverage of the artery with loss of flow to it. It can also lead to kinking of the stentgrafts which extend from the endograft to the artery if there is significant malalignment between the fenestration and ostium of the artery.

In addition, selecting branch arteries through small fenestrations may limit manipulation of angiographic catheters. Most renal arteries are transversely or cranially oriented and the mesenteric arteries are often longitudinally oriented with respect to the aorta. Selecting these many orientations through small fenestrations can be challenging due to limitations imposed on catheter maneuverability through these structures. Selection of branch arteries is made even more difficult if they have a greater than 50% stenosis as catheters must also negotiate these stenoses. Difficulties with selecting branch arteries increases procedure time which increases risk of ischemic injury to the kidneys, bowel and lower extremities.

Furthermore, because of differences in the anatomies of branch arteries from one patient to another, endograft devices with differing fenestration positions need to be tailored specifically for a patient which can require 3-4 weeks and therefore cannot be used in an emergency situation when an aneurysm ruptures.

The above limitations also occur with branched endografts which are essentially tubular extensions of fenestrations.

Another limitation of current aortic endografts is the need for large punctures into access vessels to allow for delivery of the endograft into the body. This is due to the girth of current tubular endograft devices. Even with tight constrainment of the endograft on a delivery catheter, there is a limitation on how tightly an endograft can be wound due to its tubular nature requiring 360 degrees of its fabric and metal composition to be constrained.

Active textile incorporation into an endograft which would allow the endograft or a part of the endograft to change shape could alleviate some of the limitations which exist with current endografts.

A textile is a flexible material consisting of a network of natural or artificial fibers (yarn or thread). An active textile combines smart materials with textile structure. A smart material is a material that couples two different energy domains. These energy domains include temperature, voltage, magnetic fields, and stiffness. Movement between the energy domains leads to the production of different forces and strains in the material and contributes to a complex shape change of the material.

Examples of smart materials include shape memory alloys, shape memory polymers, electroactive polymers, piezoelectric polymers, magnetorheological materials, etc. A shape memory alloy will be used as a representative smart material for the purposes of discussion. A shape memory alloy has thermo/mechanical coupling. A change in temperature of the material leads to different outputs of force and displacement. The material has a cold flexible Martensite state that can be deformed at cool temperatures. When this material is heated above its transition temperature, it undergoes a solid state phase transformation into a stiff Austenite state. This transformation is referred to as the “shape memory effect”. Therefore changes in temperature between the Martensite and Austenite states lead to reproducible changes in the shape of the material. These changes in shape can be accentuated when the smart material is incorporated into the network of a textile.

An active knit is a type of active textile. It is composed of a smart material i.e. shape memory alloy fiber that forms the unit cell of a knitted architecture composed of interlacing loops of the fiber. The internally leveraged network of unit cells that compose the active knit architecture enables complex distributed actuation motions. Active knits are capable of generating large strains beyond the base material because of their unique architecture and operation. This can lead to complex three-dimensional distributed motions.

Active knit textiles have a hierarchical architecture. The first level of the hierarchy is the knitted loop. This is the fundamental unit within a single cell in the knitted grid and leverages bending in the smart material to create larger motions. The second level is knit patterns in which the knitted loops are combined across the knitting grid in different homogeneous ways. This transforms the individual motions of the knitted loops into distributed motions. The third level includes grid patterns in which different knit patterns are combined across the knitting grid in different non homogenous ways to produce complex, non-homogeneous motions. The fourth level, restructured grids, modifies the orthogonal knitting grid into a non-planar orthogonal grid to provide complex 3 dimensional out of plane motions. The operation of active knits is an important component of creating the large complex motions provided by the knit architecture.

### SUMMARY OF THE INVENTION

Embodiments relate to an endograft device into which an active textile material is incorporated and allows the endograft to intrinsically change shape to help treat various pathologies in the body. Among the shape changes can be expansion and narrowing of the diameter, elongation and foreshortening of the length, or flowering, flaring, funneling or flattening of the ends, or other complex three dimensional out of plane motions. Embodiments also relate to shape changes occurring in components or parts of an endograft which include fenestrations, branches, gates, and limbs.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a depiction of a textile structure hierarchy according to the prior art;

FIG. 2 is a depiction of a knit architecture hierarchy according to the prior art;

FIGS. 3A and 3B depict top views of a knit loop and a purl loop, respectively according to the prior art;

FIGS. 3C and 3D depict a graphical symbols for a knit loop and a purl loop, respectively according to the prior art;

FIG. 3E depicts courses or rows and whales or columns as designated in an active knit pattern;

FIGS. 4A, 4B, and 4C depict a grid of graphical symbols of a pattern, a top view of the pattern, and a side view of the pattern, respectively according to the prior art;

FIGS. 4D and 4E depict a martensite stage of the knitted shape memory material according to the pattern of FIGS. 4A-4C, respectively according to the prior art;

FIGS. 4F and 4G depict an austenite stage of the knitted shape memory material according to the pattern of FIGS. 4A-4C, respectively according to the prior art;

FIGS. 5A, 5B, and 5C depict a grid of graphical symbols of a pattern, a top view of the pattern, and a side view of the pattern, respectively according to the prior art;

FIGS. 5D and 5E depict martensite and austenite stages of a knitted shape memory material according to the pattern of FIGS. 5A-5C, respectively according to the prior art;

FIGS. 6A-6C depict a grid of graphical symbols of a pattern, a martensite stage of the material in side view, and a austenite state of the material in side view, respectively according to the prior art;

FIGS. 7A, 7B, and 7C depict a grid of graphical symbols of a pattern, a top view of the pattern, and a side view of the pattern, respectively according to the prior art;

FIGS. 7D and 7E depict martensite and austenite stages of a knitted shape memory material according to the pattern of FIGS. 7A-7C, respectively according to the prior art;

FIGS. 8A, 8B, and 8C depict a grid of graphical symbols of a pattern, a top view of the pattern, and a side view of the pattern, respectively according to the prior art;

FIGS. 8D and 8E depict martensite and austenite stages of a knitted shape memory material according to the pattern of FIGS. 8A-8C, respectively according to the prior art;

FIG. 9A depicts a grid of graphical symbols of a pattern according to the prior art;

FIGS. 9B and 9C depict martensite and austenite stages of a knitted shape memory material according to the pattern of FIG. 9A, respectively according to the prior art;

FIG. 10A depicts a grid of graphical symbols of a pattern according to the prior art;

FIGS. 10B and 10C depict martensite and austenite stages of a knitted shape memory material according to the pattern of FIG. 10A, in an expanded and contracted state, respectively according to the prior art;

FIG. 11A depicts a grid of graphical symbols of a pattern according to the prior art;

FIGS. 11B and 11C depict martensite and austenite stages of a knitted shape memory material according to the pattern of FIG. 11A, in an expanded and contracted state, respectively according to the prior art;

FIG. 12A depicts a grid of graphical symbols of a pattern according to the prior art;

FIGS. 12B and 12C depict martensite and austenite stages of a knitted shape memory material according to the pattern of FIG. 12A, in straight and coiled states, respectively according to the prior art;

FIG. 13A depicts a grid of graphical symbols of a pattern according to the prior art;

FIGS. 13B and 13C depict martensite and austenite stages of a knitted shape memory material according to the pattern of FIG. 13A, respectively according to the prior art;

FIG. 14A depicts a grid of graphical symbols of a pattern according to the prior art;

FIGS. 14B and 14C depict martensite and austenite stages of a knitted shape memory material according to the pattern of FIG. 14A, in flat and folded states, respectively according to the prior art;

FIGS. 15A and 15B show active textile fenestrations on an endograft in the martensite stage according to an embodiment of the invention;

FIGS. 16A and 16B show active textile fenestrations on an endograft in the austenite stage according to an embodiment of the invention;

FIG. 17 depicts a juxtarenal aortic aneurysm;

FIGS. 18-24 depict steps involved in the deployment of a bifurcated abdominal aortic endograft with an active knit fenestration according to embodiments of the invention;

FIGS. 25 and 26 depict the steps involved in the deployment of an endograft contralateral extension limb through an active textile contralateral gate of a bifurcated abdominal aortic endograft according to embodiments of the invention;

FIG. 27A depicts an active textile branch in the martensite stage according to an embodiment of the invention;

FIG. 27B depicts an active textile branch in an austenite state following activation according to an embodiment of the invention;

FIG. 28 depicts the juxtarenal portion of an abdominal aortic aneurysm;

FIGS. 29-34 depict the steps involved in the deployment of the main body of an aortic endograft with active textile branches composed of two different patterns according to embodiments of the invention;

FIG. 35 depicts the gall bladder and adjacent duodenum;

FIGS. 36-42 depict the steps involved in the deployment of an active knit endograft between the gallbladder and the duodenum to create an anastomosis according to embodiments of the invention;

FIGS. 43-46 depict the steps involved in the deployment of a stretched, tightly rolled active textile sheet into the aorta and it transformation into an endograft upon activation according to an embodiment of the invention;

Of note, the right and left sides labeled on the figures depicting positions in the body in this application are done so according to the standards employed in radiological medical imaging.

Of note, the drawings and associated descriptions listed as prior art and discussed in the following detailed description are obtained from “On the role of material architecture in the mechanical behavior of knitted textiles” D Liu, DChriste, B Shakibajahromi, C Knittel, N Casteneda, D Breen, G Dion, A Kontos, International Journal of Solids and Structures Volume 109, 15 Mar. 2017 “Hierarchical architecture of active knits” J Abel, J Luntz, D Brie, Smart materials and Structures, vol. 22, 2013, “Active knit actuation architectures” J M Abel https://deepblue.lib.umich.edu/bitstream/handle/2027.42/108748, “Knitting and weaving artificial muscles” A Maziz, A Concas, A Khaldi, J Stalhand, N K Persson, E Jager, Science Advances 2017 January; 3(1). The descriptions and drawings reproduced in this application are for teaching purposes only.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

An embodiment of this proposed invention is the creation of a shape changing active textile endograft device also known as a stentgraft or covered stent composed in part or in total of a smart material configured in different textile patterns. During the manufacturing process, the smart material will be shape set to allow for transformation into at least two different shapes. The endograft will be delivered into the body in a constrained manner and unconstrained inside the body in one shape like current endografts. However unlike current endografts, it will then intrinsically undergo additional shape changes using a stimulus like temperature change or voltage. Based on the specific pattern of the active textile, the endograft or its components will change into various shapes to treat different pathologies in the body. Among the many shape changes can be expansion and narrowing, elongation and foreshortening, flowering, flaring, flattening, funneling, curling, arching, or other complex three dimensional out of plane motions. These shape changes and associated movements will allow for greater functionality than exists with current non-shape changing endograft devices.

Knitting is a traditional textile manufacturing technique that creates a network of interlacing adjacent loops that form a three-dimensional structure. The loops can be assembled into different patterns to provide different mechanical properties. In the endograft device embodiment, the device will be composed of knitted patterns of stitches of shape memory alloys such that the martensite and austenite stages of the knitted shape memory alloy will create different shapes of the device.

The material that will make up the active textile portion of the endograft device need not be a shape memory alloy. Rather, the material can be any flexible smart material knitted into a pattern such that expansion or contraction of the unit cell of the material would cause a desired change to the overall superstructure of the device.

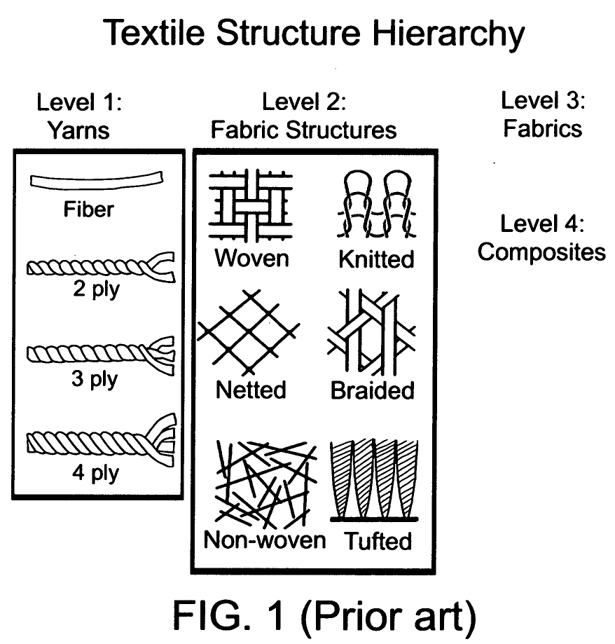

Although embodiments are described herein that are knitted, similar devices can be made using other methods depicted in the textile structure hierarchy (FIG. 1) per prior art. For example, rather than by knitting, the devices can be made using braiding, weaving, netting, etc. or a combination thereof. Furthermore, textiles can be layered and combined into different 3 dimensional shapes according to a desired plan.

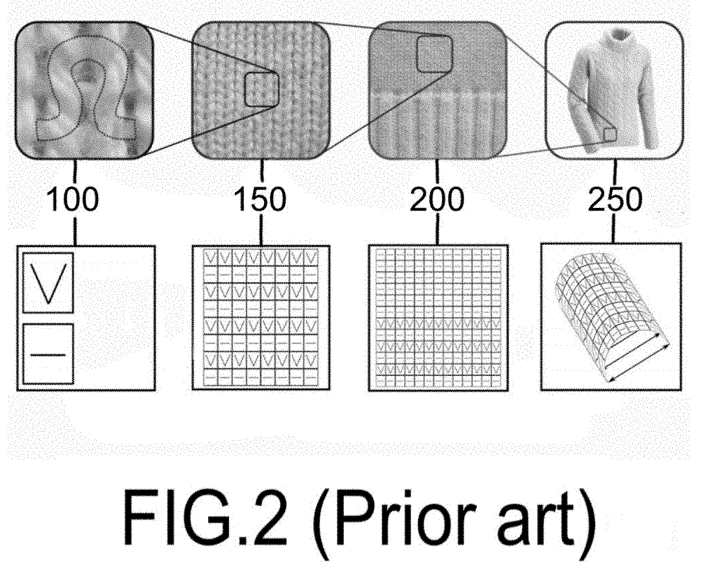

FIG. 2 depicts the hierarchical levels of knit architecture and graphical symbols for each level of the hierarchy. These levels include a knitted loop 100, knit pattern 150, grid pattern 200, and a restructured grid 250.

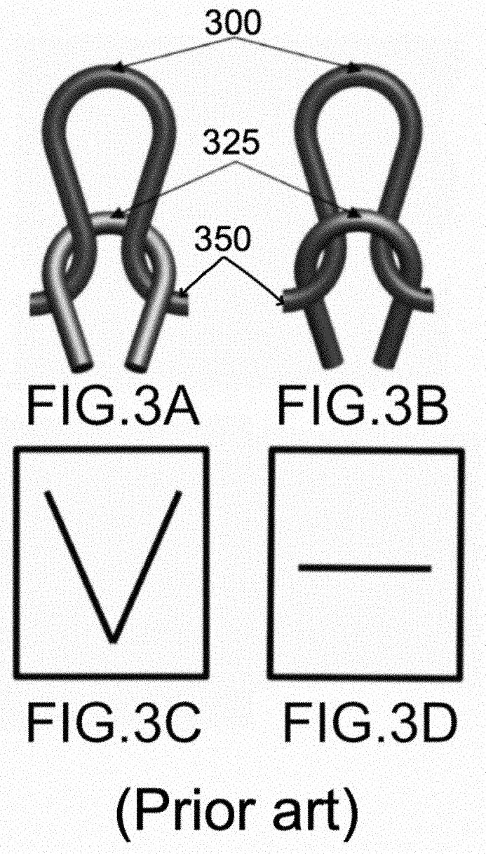

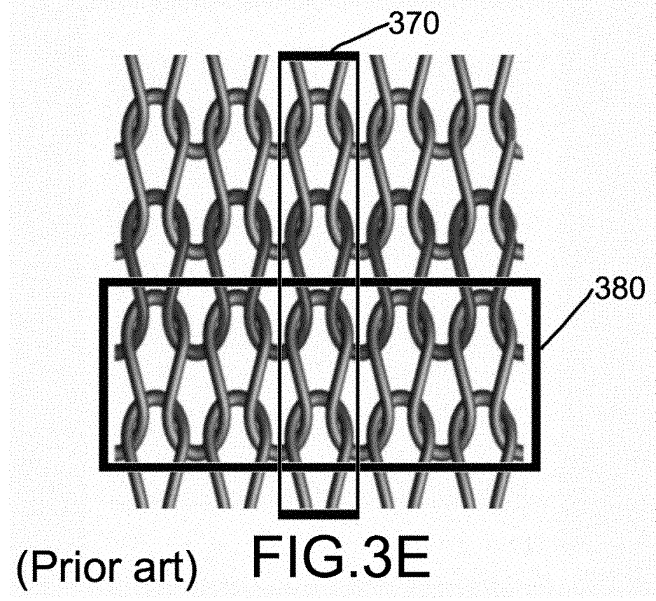

FIGS. 3A and 3B depict top views of a knit loop and a purl loop, respectively. FIGS. 3C and 3D depict graphical symbols of a knit loop and a purl loop, respectively. Various combinations of these and other knitted loops in different patterns can be used to generate the desired type of shape change in the proposed embodiment of the endograft device. FIG. 3E depicts a combination of the aforementioned loops into columns referred to as whales 370 and rows referred to as courses 380.

As shown in FIGS. 3A and 3B, knit and purl loops are unit cells made up of a loop 300, a ridge 325 and two legs 350. The loop is curved slightly more than 180°, creating a tear-drop shape that forms the upper portion of the knit unit cell. The loop extends into the legs, where adjacent unit cells attach to one another. The ridge is the uppermost portion of the loop from the previous course (row); the intersecting ridge divides the loop from the legs.

The feature that distinguishes between the loops is the location of the loop and legs with respect to the ridge. The knit loop is created by passing through the loop in the previous course from the back to the front and is characterized by a forward loop and a rear ridge. The legs of the knit loop interlock with the ridge, and then extend behind the ridge. Since the ridge is in the rear for knit loops only the base of the loop is visible; the base of the knit loop appears as a ‘V’ like shape on the textile and is represented in the symbolic grid with a ‘V’ of FIG. 3C (a standard notation in traditional knitting).

The purl loop (FIG. 3B) is created by passing through the loop in the previous course from the front to the back and is characterized by a backward loop and a forward ridge. The legs of the purl loop interlock with the ridge, and then extend in front of the ridge. Because the ridge is in the front for purl loops it is visible; the ridge of the purl loop appears as a ‘-’ like shape on the textile and is represented in the symbolic grid with a ‘-’ (a standard notation in traditional knitting—shown in FIG. 3D). A purl loop on one side of the textile appears as a knit loop on the other side.

Throughout the remainder of this application, the “V” and “-” shapes are used in the figures as shorthand to indicate knit and purl loops, respectively. Various patterns of knit and purl loops will be presented to show the variable shape changes that occur with different patterns. Some of these patterns will then be incorporated into the embodiments of the endograft device to show how the endograft can change shape to help treat different pathologies in the body.

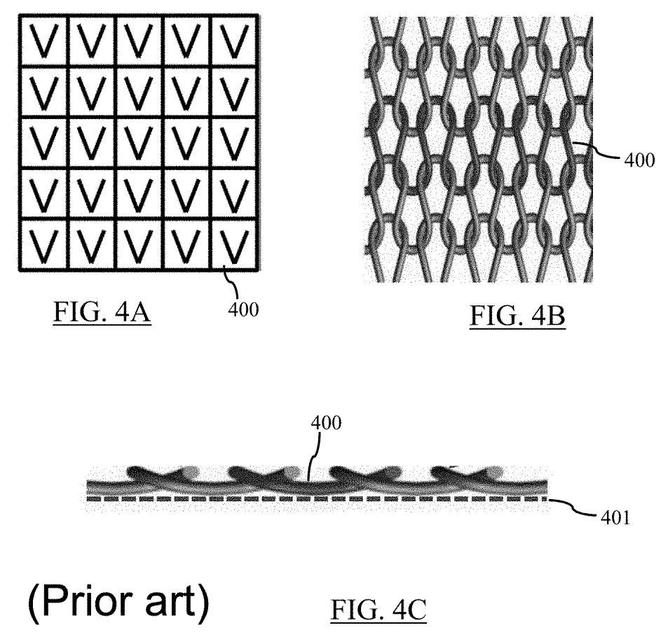

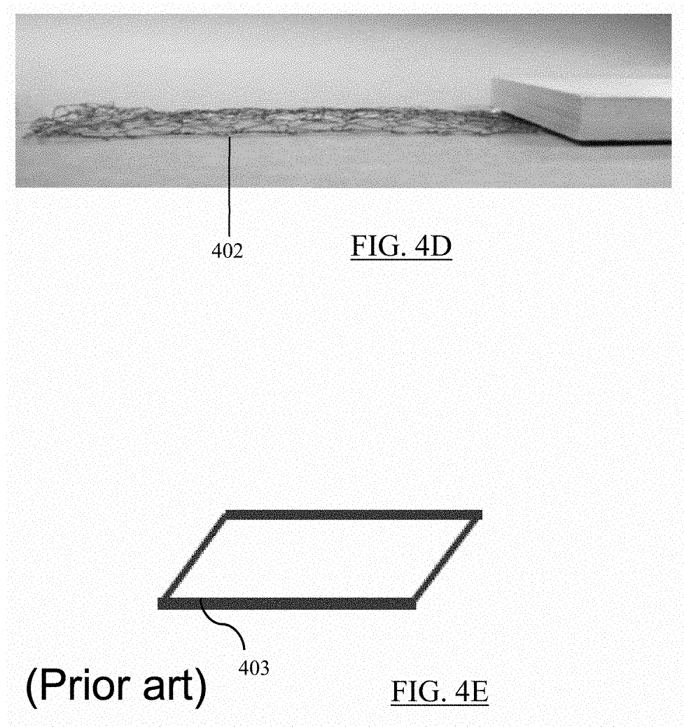

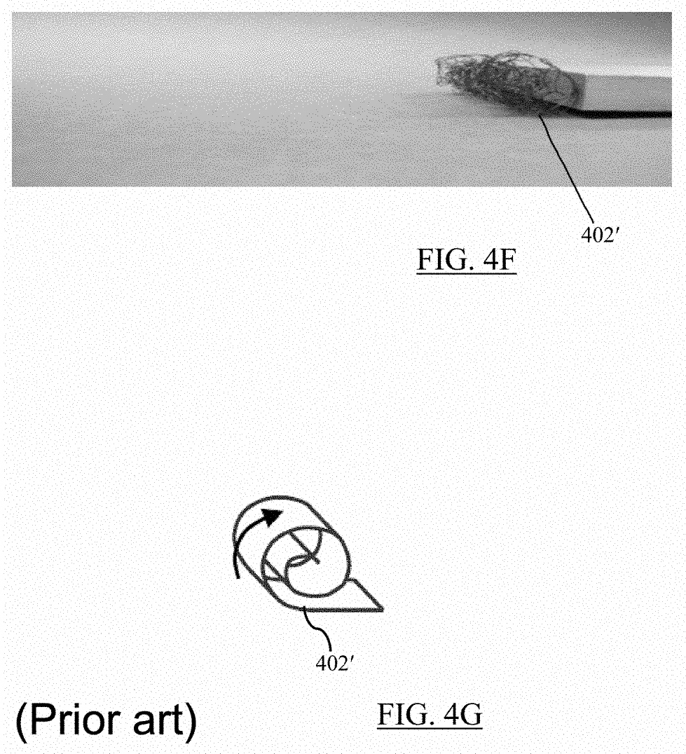

FIGS. 4A, 4B, and 4C depict a grid of graphical symbols of a pattern, a top view of the pattern, and a side view of the pattern, respectively. As shown in FIG. 4A, the pattern is entirely made of repeating rows of symbols indicating knit loops 400. FIG. 4B is a plan view of a corresponding knitted material made up of repeating rows of knit loops 400. FIG. 4C is a side view of the repeating rows of knit loops 400 previously described with respect to FIGS. 4A and 4B. As shown in FIG. 4C, the repeating rows of knit loops 400 present a smooth side 401, indicated by dashed lines. This pattern is referred to as stockinette.

FIG. 4D is a photograph of a model of a textile sheet constructed in a stockinette pattern 402 created using a smart material. This corresponds to the drawing of a rectangular stockinette sheet 403 as shown in FIG. 4E. Upon activation, which can occur when the smart material is subjected to heat, electrical current, or any other actuating force depending on the material used, the stockinette sheet rolls into a tight scroll depicted in FIGS. 4F and 4G. This change in shape represents the transition from the martensite to austenite stages of the smart material and can be utilized to create a shape change in the proposed embodiment of the endograft device.

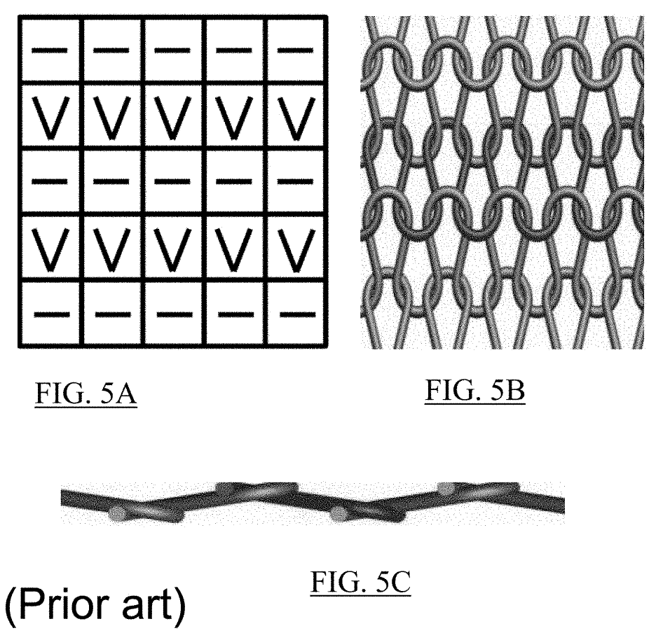

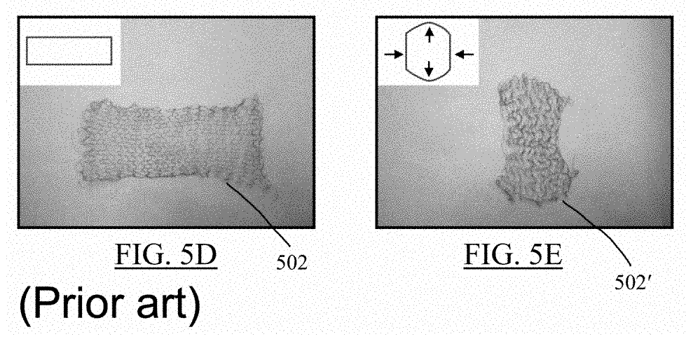

FIGS. 5A-5C depict a horizontally striped knit pattern called a garter composed of alternating rows of knit and purl loops. FIGS. 5A, 5B, and 5C depict a grid of graphical symbols of the pattern, a top view of the pattern, and a side view of the pattern, respectively. FIG. 5D is a photograph of a model of a garter textile sheet 502 created using a smart material. Upon activation, the garter sheet contracts in width while expanding vertically to form a narrower sheet depicted in FIG. 5E 502′.

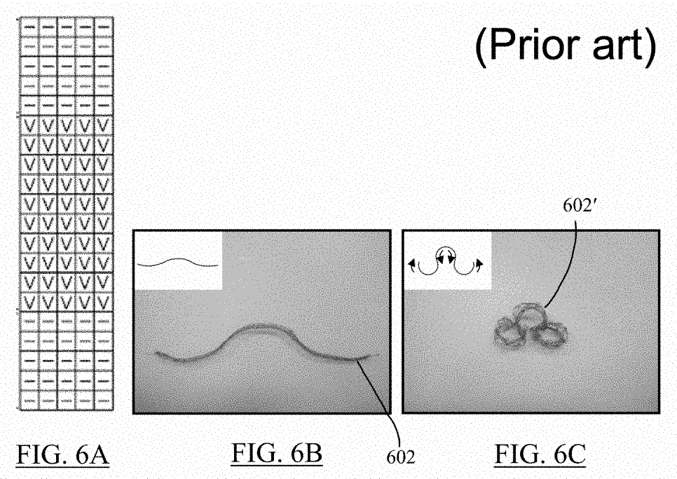

FIG. 6A depicts a grid of graphical symbols of a welt pattern. FIGS. 6B and 6C depict a textile model of a welt pattern knitted from a smart material. FIGS. 6B and 6C show the shape change that occurs with this pattern upon activation. FIG. 6B shows a relaxed welt 602 in the side view before activation and FIG. 6C shows a curled welt 602′ in side view following activation. Upon activation, the welt pattern contracts and curls into 3 loops.

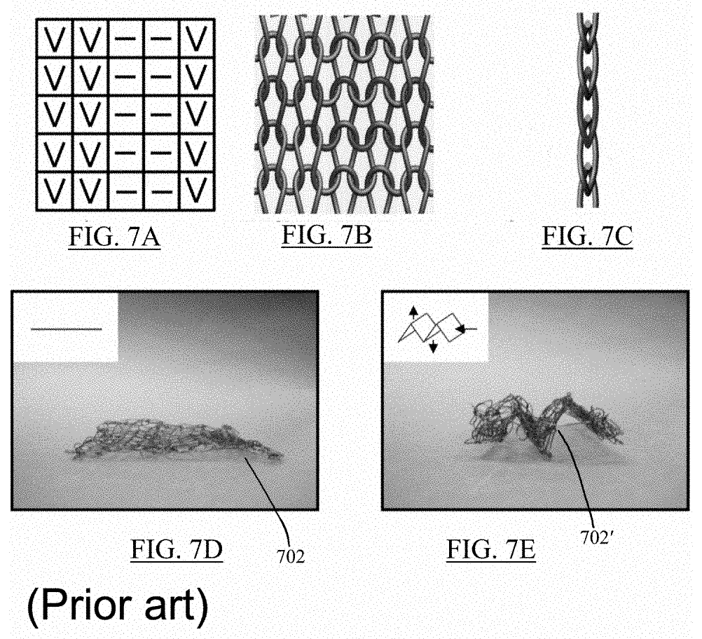

FIGS. 7A, 7B, and 7C depict a grid of graphical symbols of a vertically striped or rib knit pattern, a top view of the pattern, and a side view of the pattern, respectively. FIG. 7D depicts a relaxed vertically striped knit model 702 before activation and FIG. 7E depicts a curled vertically striped knit model 702′ following activation. Upon activation, the rib knit pattern accordions to form ridges.

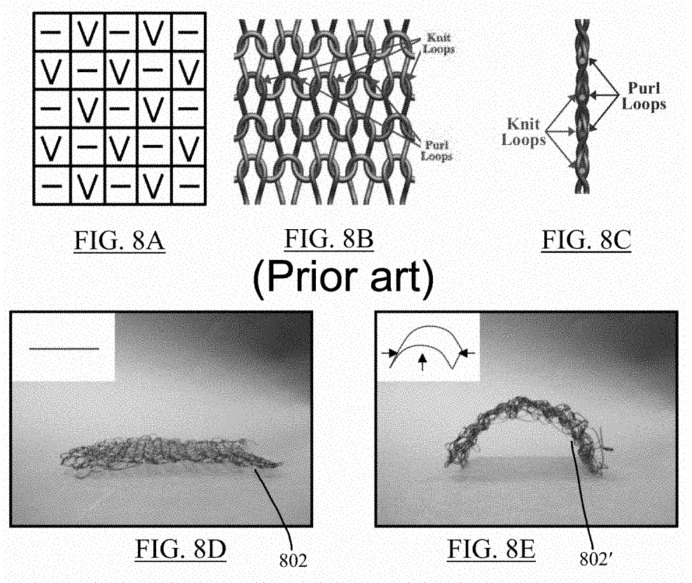

FIGS. 8A, 8B, and 8C, depict a grid of graphical symbols of a diagonally striped or seed pattern, a top view of the pattern, and a side view of the pattern, respectively. FIG. 8D depicts a relaxed diagonally striped knit model 802 before activation, and FIG. 8E depicts a curled diagonally striped knit model 802′ following activation. Upon activation, the seed stitch pattern forms an arch.

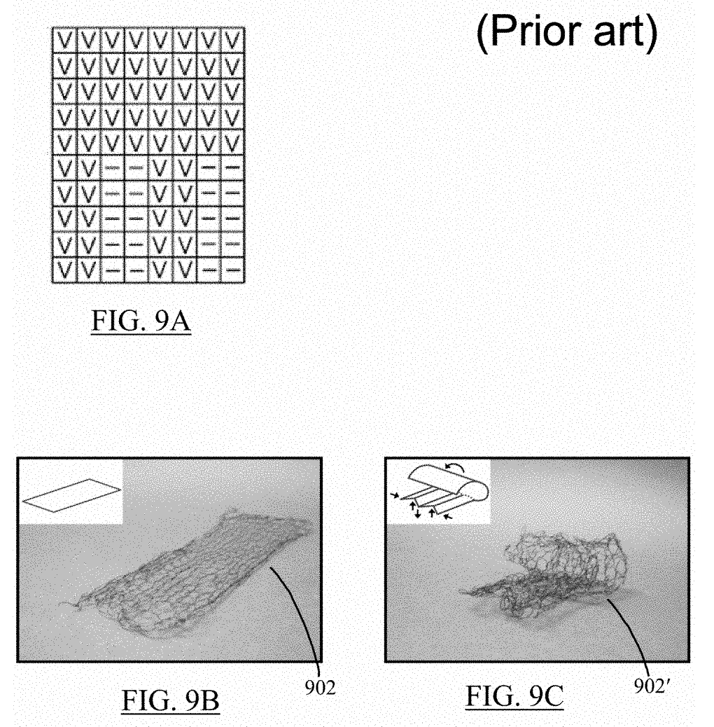

FIG. 9A depicts a grid of graphical symbols of a pattern composed of two different knit patterns. This pattern represents a grid pattern 200 in the knit hierarchy in FIG. 2. As shown in FIG. 9A, the top five rows are a stockinette pattern and the bottom five rows are a rib knit pattern. FIG. 9B depicts a relaxed model 902 of the pattern depicted in FIG. 9A before activation and FIG. 9C depicts a partially curled and contracted model 902′ following activation. Upon activation, half of the textile contracts and the other half flips over to cover the contracted portion. This pattern shows that more than one shape motion can be shape set into a single textile. This can allow for different kinds of shape changes in different parts of a single endograft device.

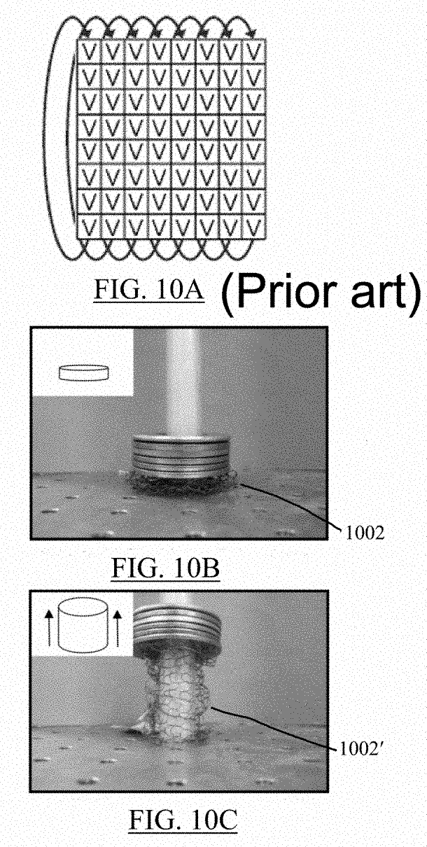

FIG. 10A depicts a grid of graphical symbols of a pattern in the stockinette configuration as described earlier. In contrast to the other knit patterns shown and described above, the knit pattern shown in FIG. 10A is circularized and forms a full cylindrical loop. FIGS. 10B and 10C depict martensite and austenite stages of a knitted shape memory material model according to the pattern of FIG. 10A, in a compact and an expanded state 1002 and 1002′, respectively. These occur before and after activation. FIGS. 10B and 10C show how activation of this knitted pattern can narrow and elongate the cylindrical loop. This pattern represents a restructured grid 250 in the knit hierarchy FIG. 2. Activation of this pattern shows how an endograft or a branch of an endograft constructed of this architecture can narrow and elongate or widen and foreshorten.

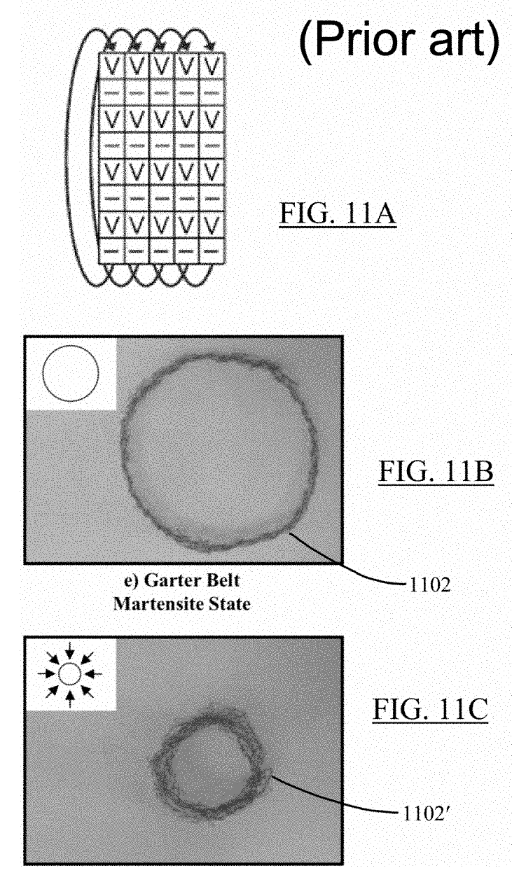

FIG. 11A depicts a grid of graphical symbols of a pattern in the garter belt configuration as described earlier. Similar to the knit pattern shown in FIG. 10A, this pattern is also circularized to form a full cylindrical loop. FIGS. 11B and 11C depict martensite and austenite stages of a knitted shape memory material model according to the pattern of FIG. 11A, in an expanded and narrowed state 1102 and 1102′, respectively. These occur before and after activation. FIGS. 11B and 11C show how activation of this knitted pattern can expand and narrow the cylindrical loop. This pattern also represents a restructured grid 250 in the knit hierarchy FIG. 2. Activation of this pattern shows how an endograft or a fenestration of an endograft constructed of this architecture can expand or narrow.

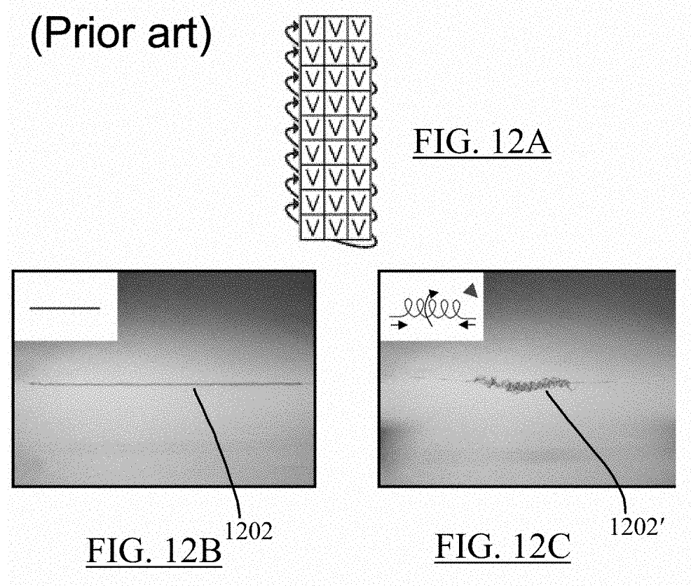

In addition to the circularization of patterns as depicted in FIGS. 10A-C and 11A-C, patterns can also be restructured by helically knitting across the textile to form a knitted tube. This is referred to as course-wise restructuring and its graphical symbol is depicted in FIG. 12A. The pattern created by this type of restructuring forms what are called I-cord textiles. FIGS. 12B and 12C depict martensite and austenite stages of a knitted shape memory material composed of the pattern depicted in FIG. 12A, in straight and coiled states, respectively. As shown in FIGS. 12A-12C, courses of a stockinette grid pattern can be restructured by helically knitting across the textile to form a knitted tube which changes shape from a thin long tube 1202 into a helically coiled shape 1202′, following activation. Course-wise restructuring forms restructured grids that can produces novel out-of-plane actuation behavior.

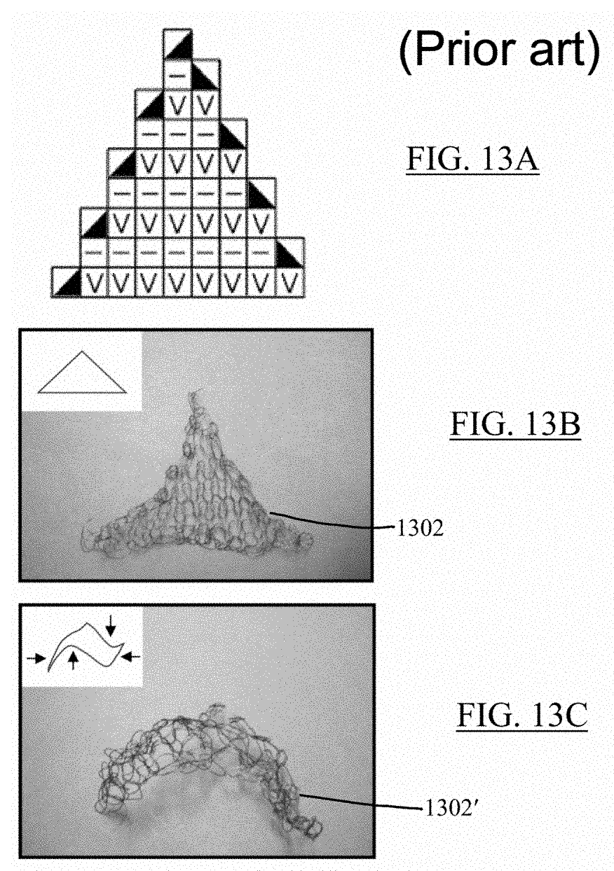

In addition to the restructured grids shown above, adjacent grid cells can be merged together. This decreases the number of loops in a course, creating a restructured grid that results in textiles with complex non rectangular shapes. FIG. 13A depicts a grid of graphical symbols of a pattern. In addition to knit and purl identifiers, FIG. 13 also includes indications to merge (i.e., “knit together” or “purl together”) adjacent stitches in alternating courses to form a triangularly shaped model 1302 as shown in FIG. 13B. Activation of the memory shape alloy causes the model to deform and become a contracted triangle shape 1302′. The curl is in opposite directions at each edge due to the asymmetric boundary conditions in the austenite state.

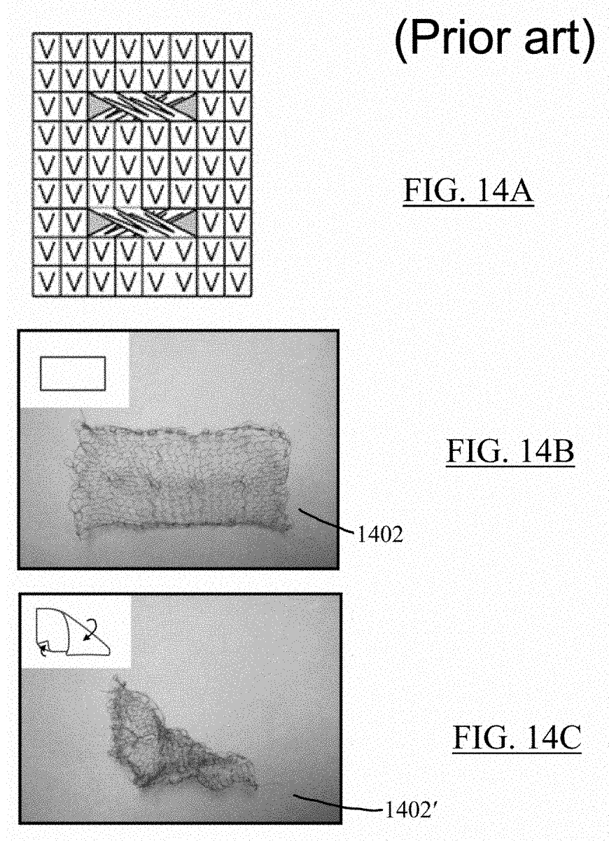

FIG. 14A depicts a grid of graphical symbols of a pattern according to another restructured grid referred to as a reordered grid. In reordered grids loops are crossed or stretched into different columns or rows. FIG. 14A shows a reordered grid similar to that used to make cables in a knitted textile in which the four inner most knit cells are overlapped. The two cells adjacent to the overlapped cells on the right are bent and stretched to the left and the two cells adjacent to the overlapped cells on the left are bent and stretched to the right. FIGS. 14B and 14C depict martensite and austenite stages 1402 and 1402′ of a knitted shape memory material model according to the pattern of FIG. 14A. As shown in FIG. 14C, following activation, the model 1402′ is torqued along its length causing it to curl about its vertical axis. Re-ordering the grid creates new motions during activation of the shape memory material textiles that are unavailable with knit patterns alone.

The above examples of active knit architectural patterns encompassing the spectrum of the knit hierarchy are meant to show wide shape changing potential that exists with current active textiles. There are many additional active textile patterns that can be created using variable positioning and connections of knitted loops, knit patterns, grid patterns, and restructured grids. Moreover, these patterns can be additionally varied by changing the loop height, width, length, diameter, and shape. This allows for a multitude of differing shape changes that can occur based on the variable textile architecture. In such a manner, different active textile architectural patterns can be incorporated into the structure of an endograft including its components to achieve multitudes of shape changes that would help in the treatment of varying pathologies inside the body.

One embodiment is a fenestrated endograft (stentgraft) in which active textile material is incorporated into the fenestration of the graft. The fenestrations are configured for example in a cylindrical garter belt pattern or as a yarn incorporated as a pursestring around a fenestration. Initially the fenestrations are very large in a relaxed martensite stage. They do not require exact positioning or alignment with branch vessels that need to be selected for preservation. Large fenestrations also allow for easier angiographic catheter manipulation to gain access into branch vessels.

Once a branch vessel is selected, a stentgraft or vascular balloon is deployed inside the vessel to protect the intraluminal space of the vessel. The stentgraft or balloon is extended through the fenestration into the endograft. The garter belt patterned active knit fenestrations is then activated to undergo a shape change into the austenite stage in which the diameter of the fenestration decreases and it tightens around the stentgraft or balloon to form a tight seal. This allows for blood flow from the endograft directly into the branch vessel and not into the aneurysm sac.

In this embodiment, a balloon is preferable in situations where there is good wall apposition by the endograft with the aortic wall immediately adjacent to or surrounding the ostium or opening of the branch vessel. This precludes the need for leaving behind a stentgraft from the endograft to the branch vessel. This also prevents future complications such as stent thrombosis or intimal hyperplasia contributing to an edge of stent stenosis. Alternatively, stentgrafts may be preferable when there is not good wall apposition by the endograft with the aortic wall immediately adjacent to or surrounding the ostium of the branch vessel.

The embodiment described above is depicted in the following figures. Of note, the right and left sides labeled on all of the following figures are done so according to the standards employed in radiological medical imaging.

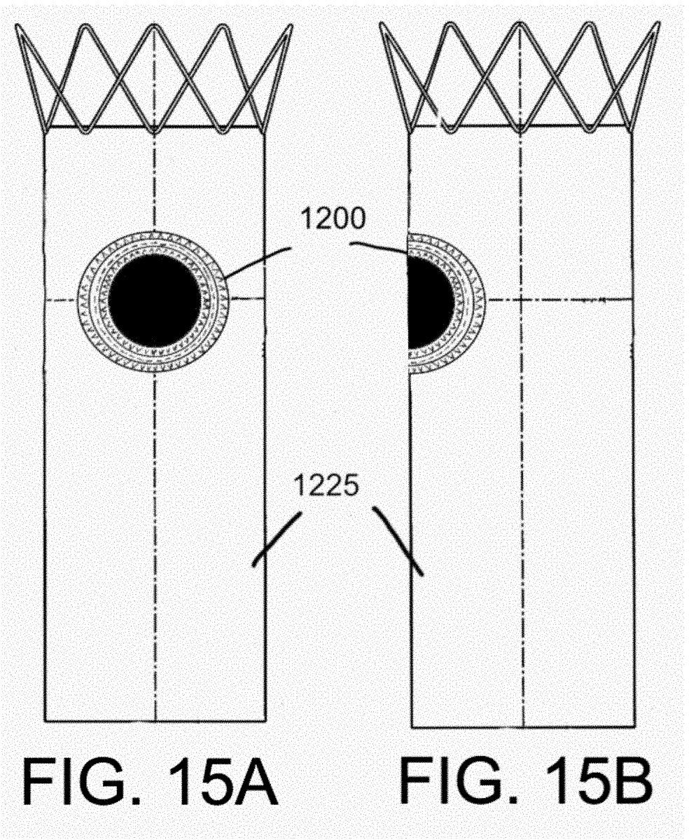

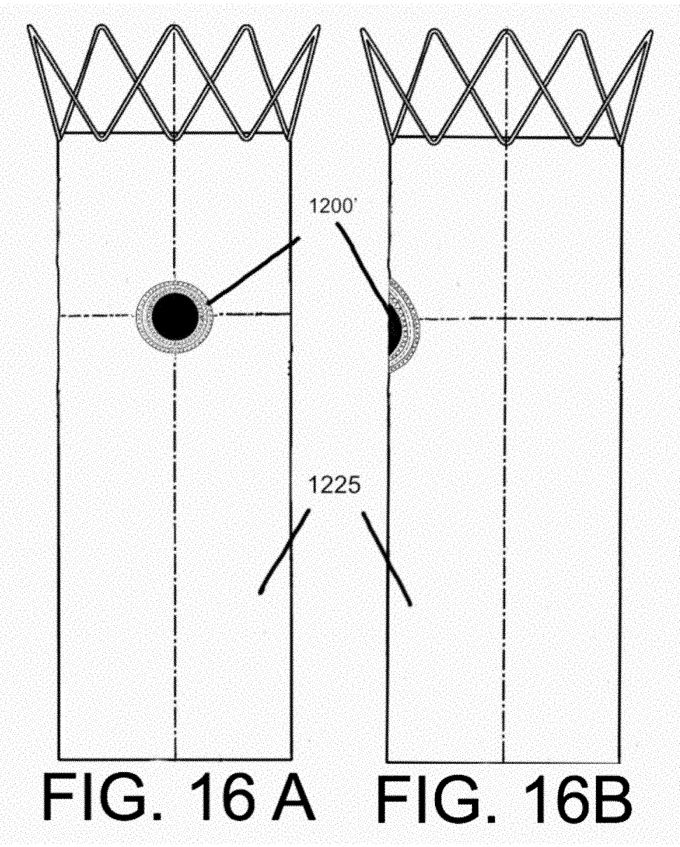

FIGS. 15A and 15B show active textile fenestrations 1200 configured in a cylindrical garter belt type pattern FIG. 11A and located on the main body of a tubular endograft 1225. FIG. 15A shows a frontal view of the fenestration and FIG. 15B shows a side view of the fenestration on the endograft. The fenestrations are in a relaxed martensite stage similar to the model depicted in FIG. 11B 1102.

FIGS. 16A and 16B show the active textile fenestrations 1200′ constructed in a cylindrical garter belt type pattern in a narrowed austenite stage post activation similar to the model depicted in FIG. 11C 1102′. FIGS. 16A and 16B show the fenestrations in the frontal and side views respectively.

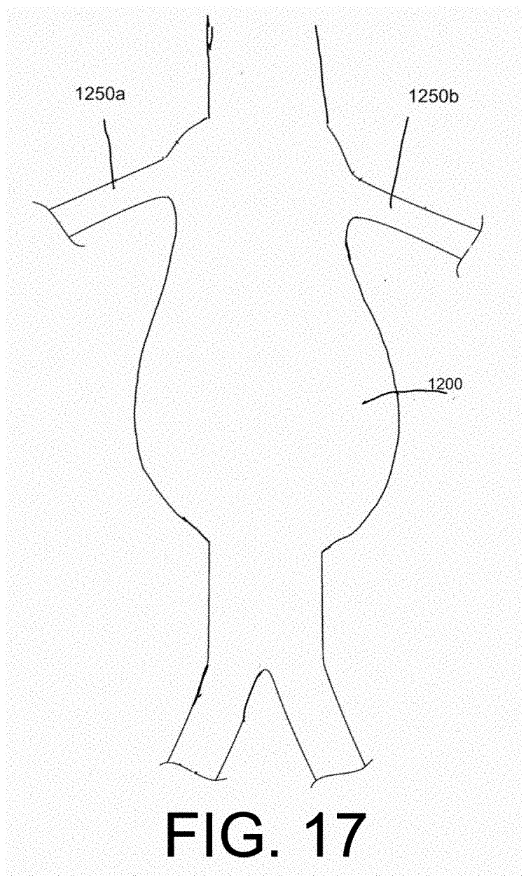

FIG. 17 is a depiction of a juxtarenal abdominal aortic aneurysm 1200. The aneurysm extends across the origins of the right and left renal arteries 1250 a and 1250b listed respectively.

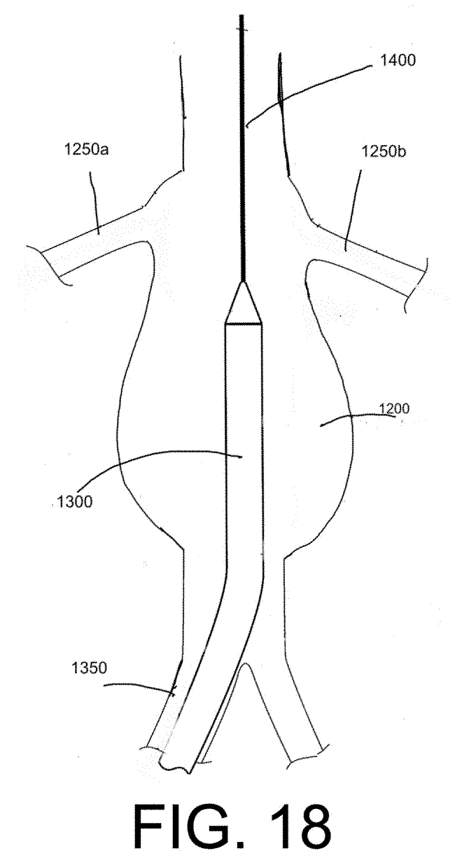

FIG. 18 depicts the passage of an endograft delivery catheter 1300 into the abdominal aortic aneurysm 1200 via the right common iliac artery 1350 over a guidewire 1400.

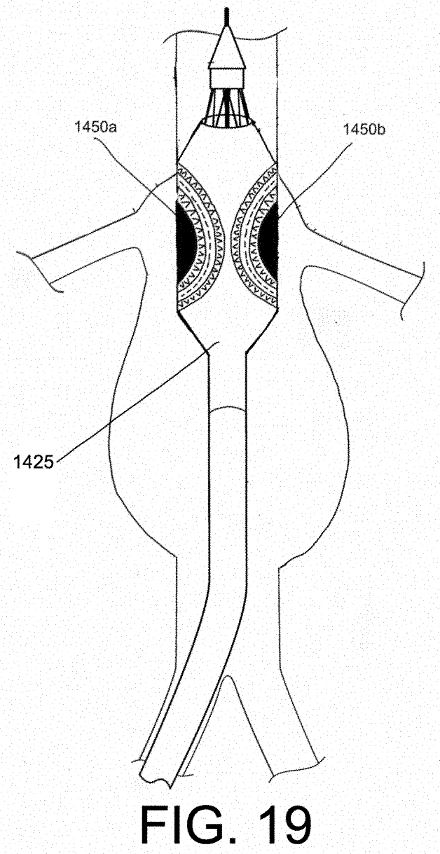

FIG. 19 depicts unsheathing and partial deployment of the main body of an endograft 1425 with large active textile fenestrations constructed in a cylindrical garter belt type pattern 1450a and 1450b in the juxtarenal portion of the abdominal aorta.

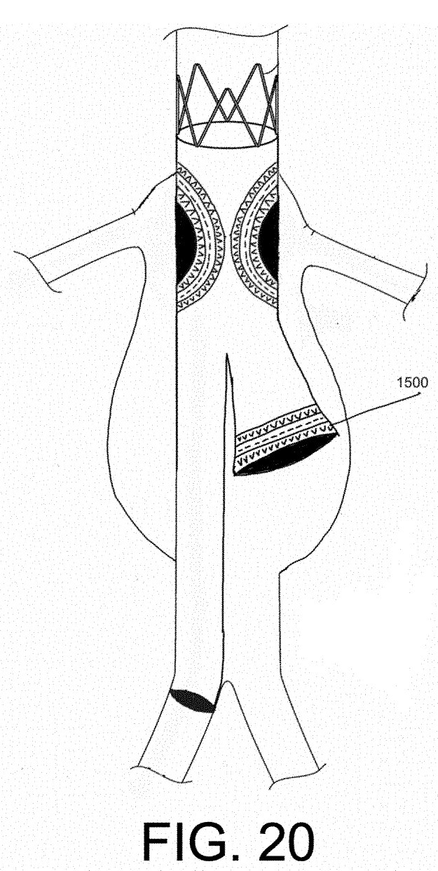

FIG. 20 depicts complete deployment of the bifurcated abdominal aortic endograft with large active textile fenestrations and an active textile configured contralateral gate 1500 constructed in a cylindrical garter belt type pattern.

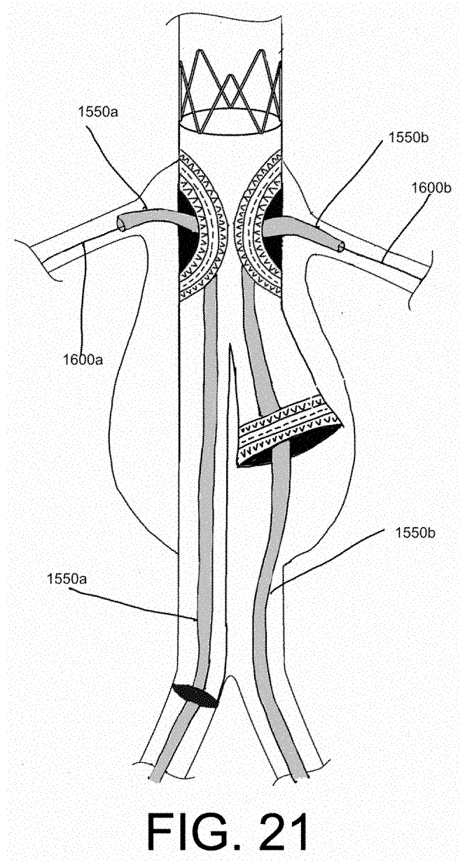

FIG. 21 depicts selection of the renal arteries through the active textile fenestrations by angiographic catheters 1550a and 1550b passed through both limbs for passage of guidewires 1600a and 1600b into the renal arteries.

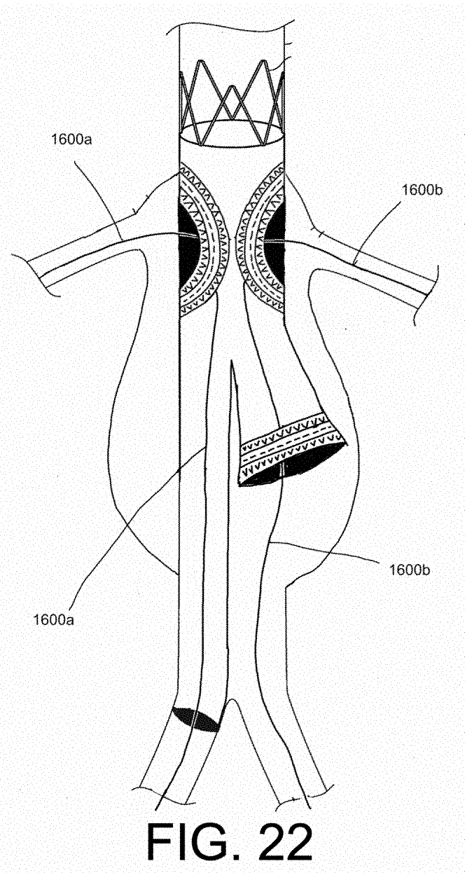

FIG. 22 depicts the removal of the angiographic catheters leaving behind guidewires 1600a and 1600b which extend from the right and left iliac arteries into the renal arteries through the active textile fenestrations.

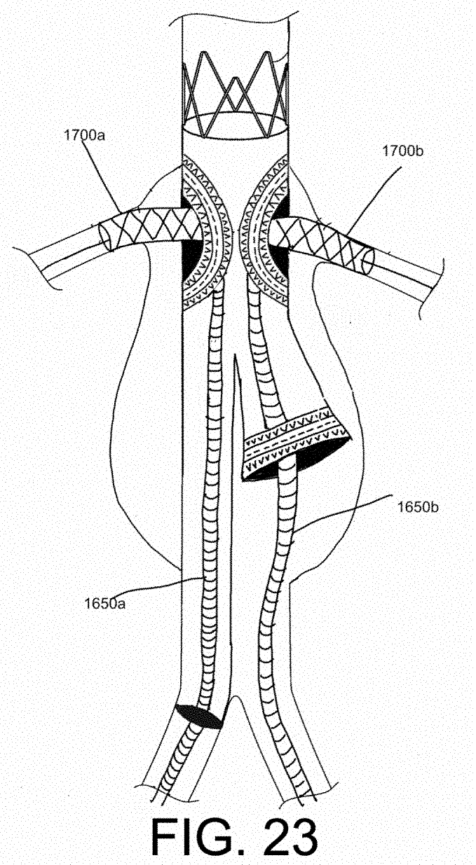

FIG. 23 depicts the passage of delivery catheters 1650a and 1650b for stentgrafts 1700a and 1700b over the guidewires across the active textile fenestrations of the endograft into the renal arteries. The stents are deployed in the renal arteries and extended across the active textile fenestrations.

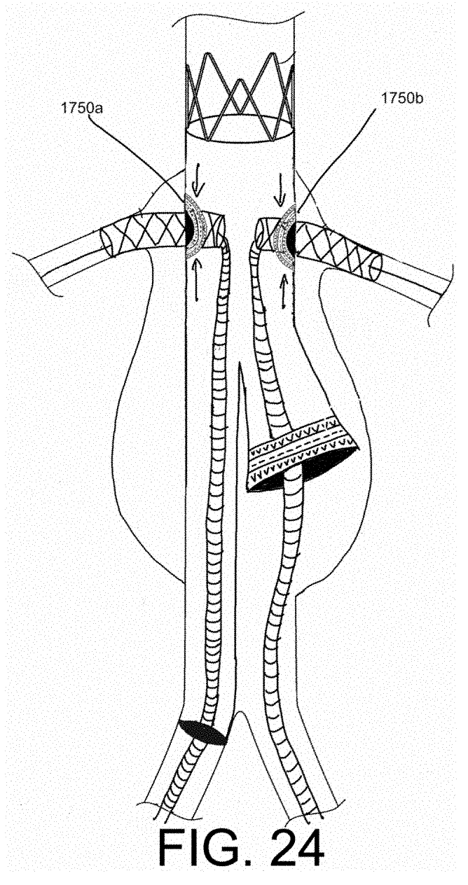

FIG. 24 depicts activation of the active textile fenestrations 1750a and 1750b into the austenite stage which leads to narrowing of the lumen of the fenestrations around the deployed stents to create a tight seal. The narrowing of the fenestrations is depicted by arrows and is similar to the shape change depicted in FIGS. 11A and 11B. The seal between the fenestration and stents allows for passage of blood from the aortic endograft into the renal arteries without leakage into the aneurysm sac.

A second embodiment is the incorporation of active textile material in the contralateral gate of a bifurcated endograft. Current abdominal aortic endografts usually consists of a bifurcated main body with an ipsilateral limb extending into an ipsilateral iliac artery and a contralateral gate that opens in the aneurysm sac. An angiographic catheter introduced from the contralateral iliac artery is used to select the contralateral gate to allow for passage of a guidewire through the contralateral gate. Over the guidewire a contralateral endograft limb is passed from the contralateral iliac artery and deployed through the contralateral gate into the bifurcated endograft to complete construction the bifurcated endograft with two limbs extending into each iliac artery.

Due to a small diameter of the contralateral gate on existing bifurcated endografts, it can be challenging to select the contralateral gate with an angiographic catheter especially when the small diameter gate is floating in a great expanse of space in a large aneurysm sac. Therefore for the proposed embodiment, the contralateral gate would be composed of an active textile material constructed in a cylindrical garter belt type pattern to create a large flared, funnel shape. This would make its selection by a catheter easier. Once the contralateral endograft limb has been deployed across the contralateral gate, the gate would be activated and tightened around the limb much like the cylindrical garter belt patterned fenestration illustrated earlier.

The following figures describe the second embodiment.

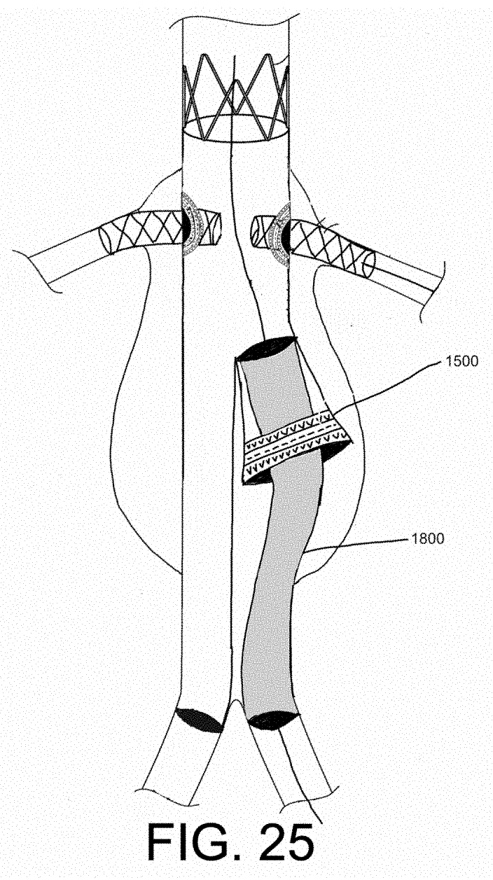

FIG. 25 depicts the passage of a contralateral endograft limb 1800 through the active textile cylindrical garter belt configured contralateral gate 1500 of the bifurcated abdominal aortic endograft.

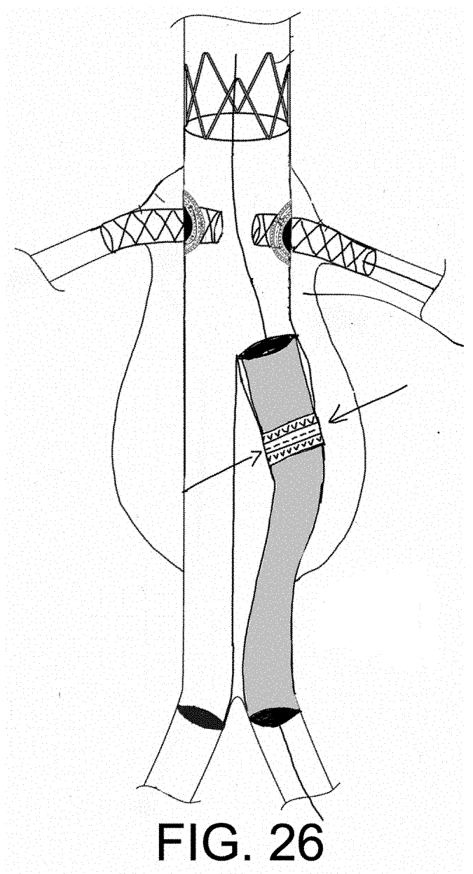

FIG. 26 depicts activation of the active textile configured contralateral gate of the bifurcated abdominal aortic endograft into the austenite stage which leads to narrowing of the lumen of the gate around the contralateral endograft limb to create a tight seal. The shape changing motion is depicted by arrows. This allows for passage of blood from the bifurcated aortic endograft into the iliac arteries and completely excludes the abdominal aortic aneurysm from arterial blood flow.

A third embodiment is a branched endograft in which active textile material is incorporated into the branches of the endograft. The branches are configured for example in a cylindrical stockinette belt pattern FIG. 10A. Initially they are foreshortened with a large diameter in a relaxed martensite stage similar to the model shown in FIG. 10B. They do not require exact positioning or alignment with branch vessels that need to be selected for preservation. The large diameter allows for easier angiographic catheter manipulation to gain access into branch vessels.

Once a branch vessel is selected with a guidewire, a vascular balloon is extended through the endograft branch over the guidewire and deployed inside the vessel to protect the intraluminal space of the vessel. The stockinette belt patterned active knit branch is then activated to undergo a shape change into the austenite stage in which the diameter of the branch decreases and it elongates and extends to the ostium of the branch vessel over the balloon. This shape change is similar to the model shown in FIG. 10C.

The end of the branch graft is constructed of a different active textile pattern similar to a grid pattern in the hierarchy of knit architecture. When activated it forms a flattened, flared, round, or funnel type of shape to better appose the vessel wall around the ostium of the branch vessel. Endoanchors/barbs/screws may be incorporated onto the flared portion of the elongated endograft branch to help secure it around the ostium of the branch vessel and achieve seal. In this manner a stent would not need to be left behind in the branch vessel. When there is poor wall apposition, a stent would be left behind extending from the lumen of the artery into the endograft branch.

The third embodiment described above is depicted in the following figures:

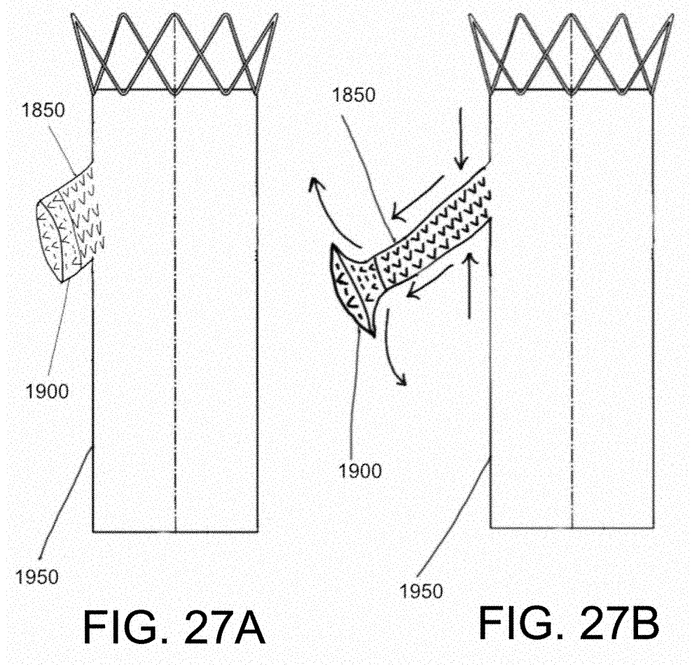

FIGS. 27A and 27B show active textile branches arising from the main body of an endograft 1950b shown in a side view. The branches are composed of two different active knit patterns. The portion of the branch closest to the main body (proximal portion) 1850 is composed of a cylindrical stockinette pattern. The portion of the branch furthest from the main body (distal portion) 1900 is composed of separate pattern in a restructured grid type architecture. FIG. 27A depicts the active textile branch in the martensite stage. FIG. 27B depicts the active textile branch in an austenite state following activation. Upon activation the proximal portion 1850 narrows and elongates while the distal 1900 flowers outward and flattens. The shape changing motions are depicted by arrows in FIGS. 27A and 27B.

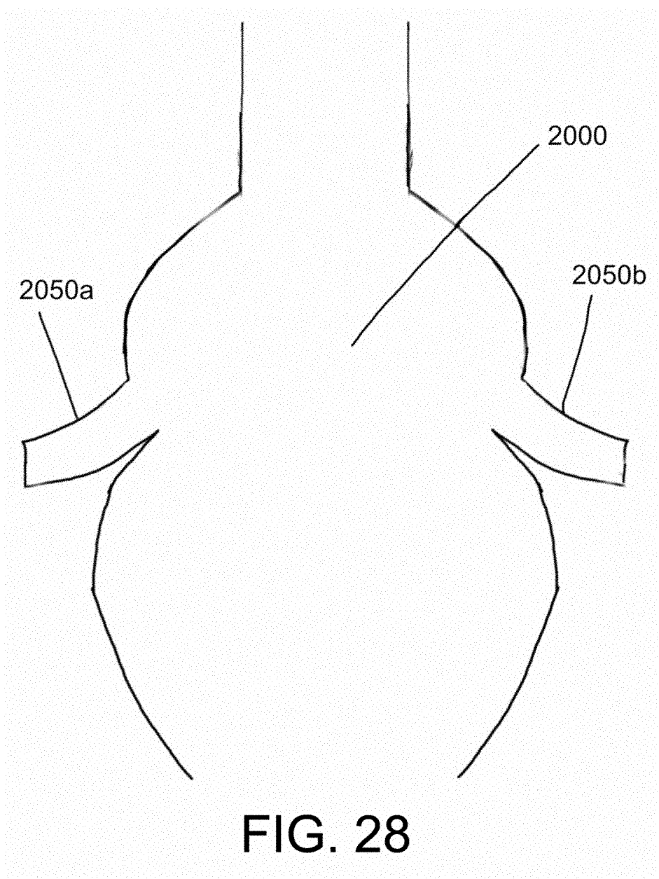

FIG. 28 depicts the juxtarenal portion 2000 of an abdominal aortic aneurysm with the right 2050a and left 2050b renal arteries respectively.

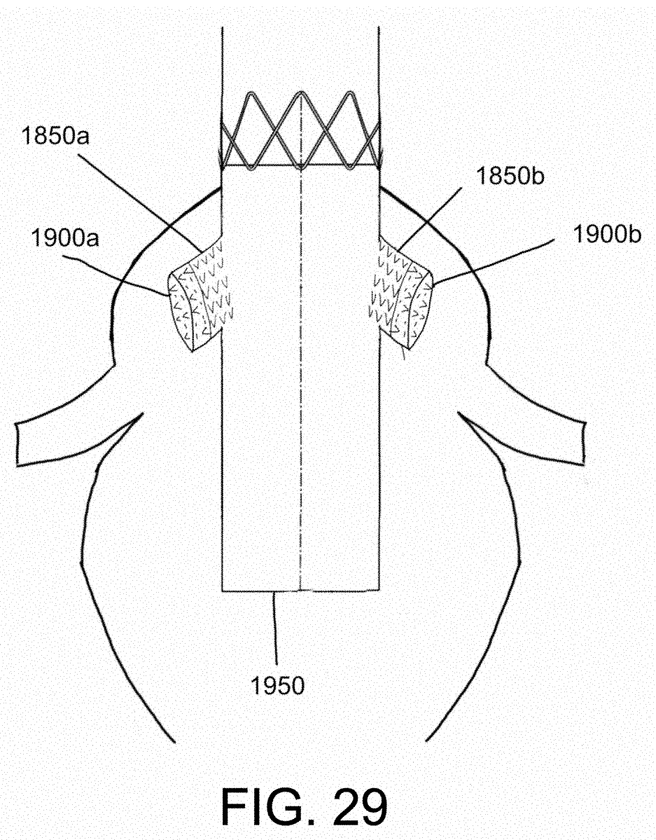

FIG. 29 depicts the deployment of the main body of an aortic endograft 1950 into the juxtarenal portion of an abdominal aortic aneurysm. The main body has active textile branches composed of two different active knit patterns as described in FIGS. 27A and 27B. The patterns on the right are labeled 1850a and 1900a and the patterns on the left are labeled 1850b and 1900b.

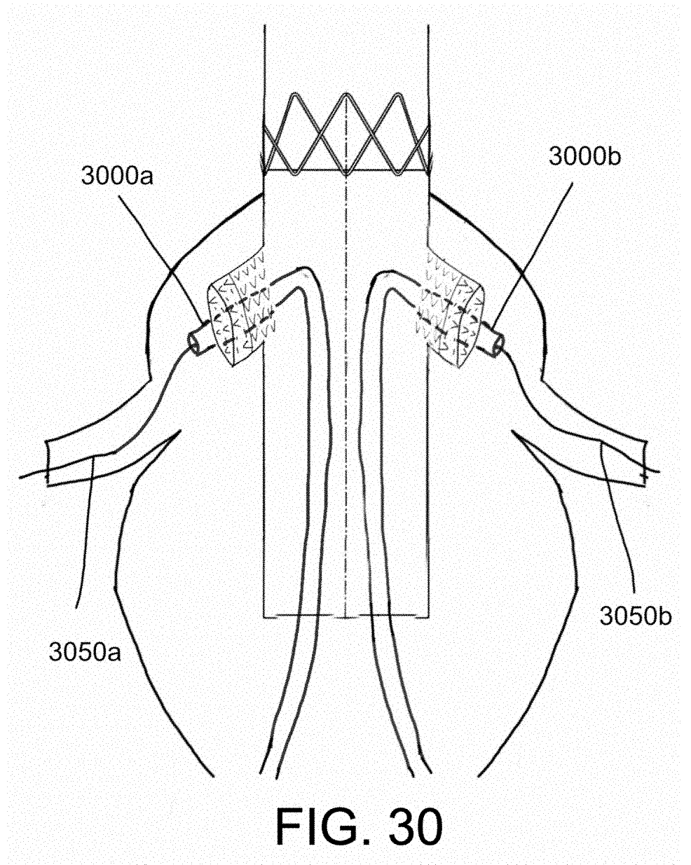

FIG. 30 depicts the selection of active textile branches by angiographic catheters 3000a and 3000b for deployment of guidewires 3050a and 3050b into the renal arteries.

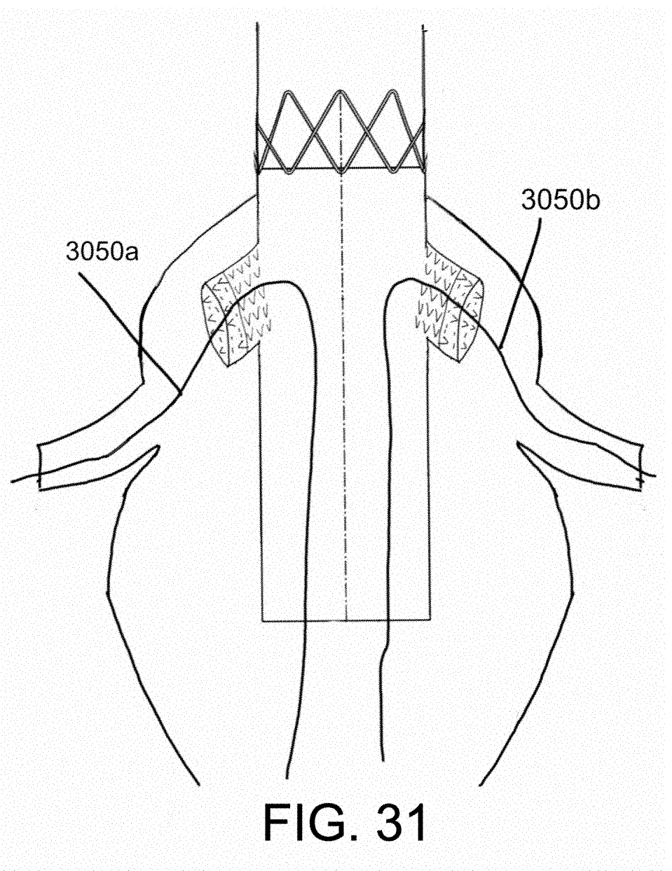

FIG. 31 depicts removal of the angiographic catheters with guidewires 3050a and 3050b left behind in the renal arteries across the active textile branches.

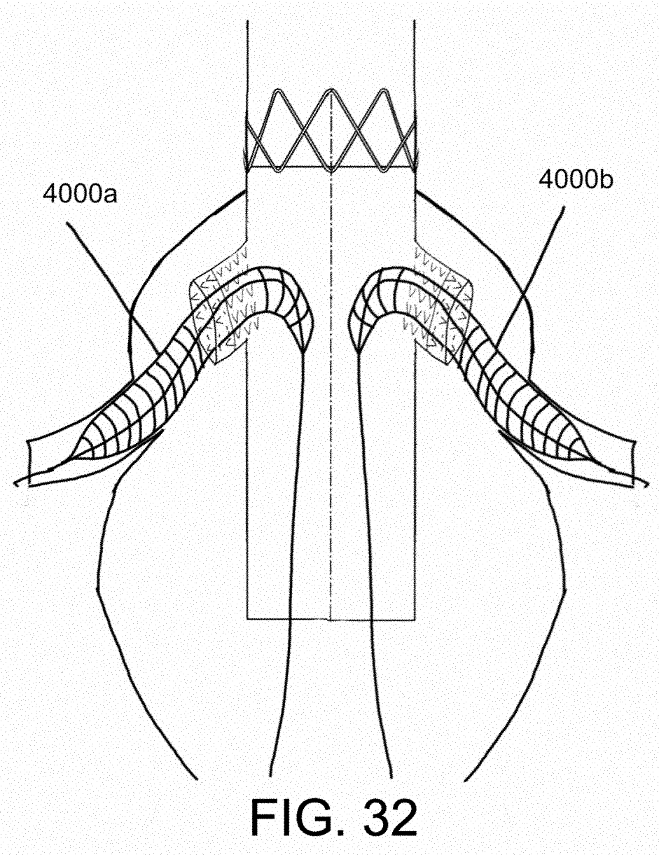

FIG. 32 depicts passage of vascular balloons 4000a and 4000b over the guidewires, through the active textile branches and into the renal arteries.

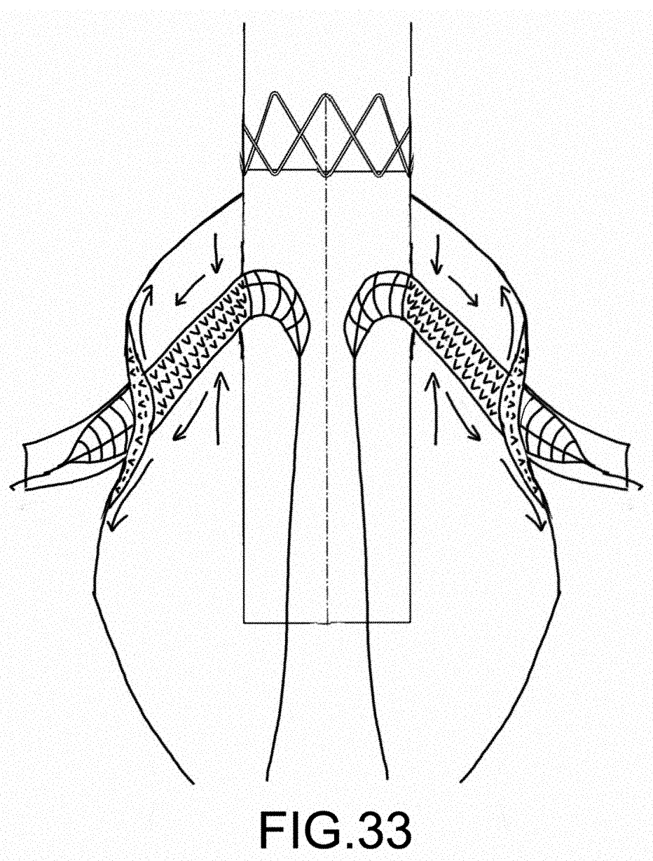

FIG. 33 depicts activation of the active textile branches with narrowing and elongation of the proximal portion and flaring, flowering/flattening of the distal portion around the vascular balloons and around the ostia of the renal arteries. The shape changing motions are depicted by the arrows in the FIG. 33 and correspond to the different patterns on the proximal and distal portions of the active textile branches.

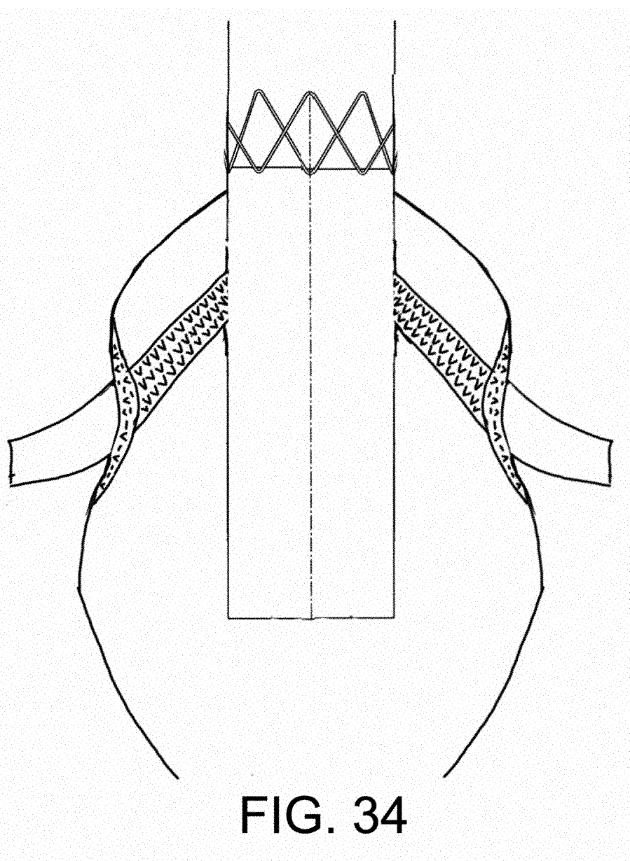

FIG. 34 depicts the final position of the active textile branches post activation after balloon removal extending from the main body and completely apposing the wall of the aorta around the ostia of the renal arteries to create a tight seal.

A fourth embodiment is an active textile endograft which creates an anastomosis between two tubular or hollow organs. In such an embodiment, the endograft is placed in between the two organs and activated. Upon activation, the two ends of the endograft flare, flower, flatten outward and the middle portion foreshortens. The shape change of the ends creates wall apposition between the ends and the adjacent tissue of the inner wall of the hollow organ. The shape change of the middle pulls the two organs together and creates a channel between them. This embodiment can also be utilized to create anastomotic type connections between one or more endografts.

The fourth embodiment described above is depicted in the following figures:

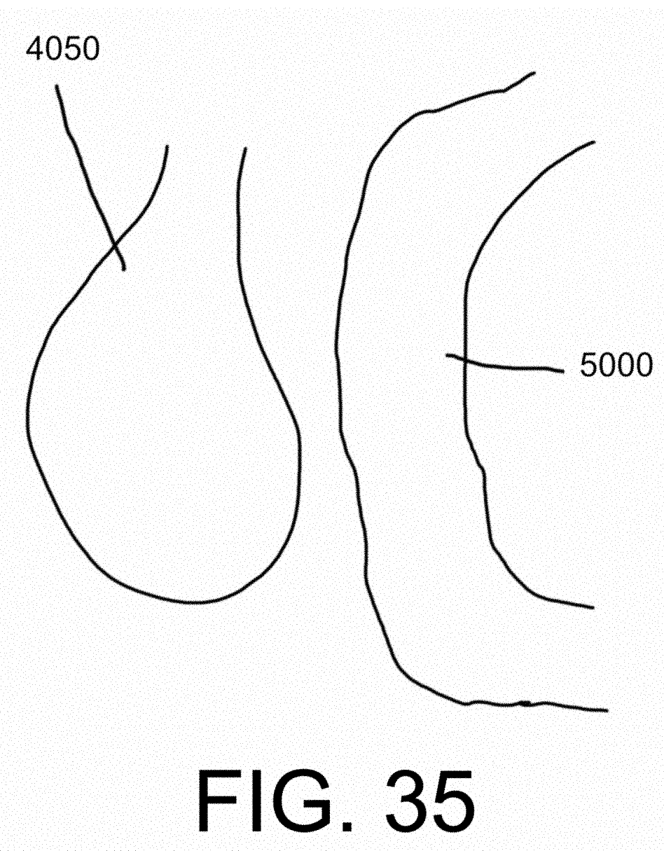

FIG. 35 shows the gallbladder 4050 and adjacent duodenum 5000.

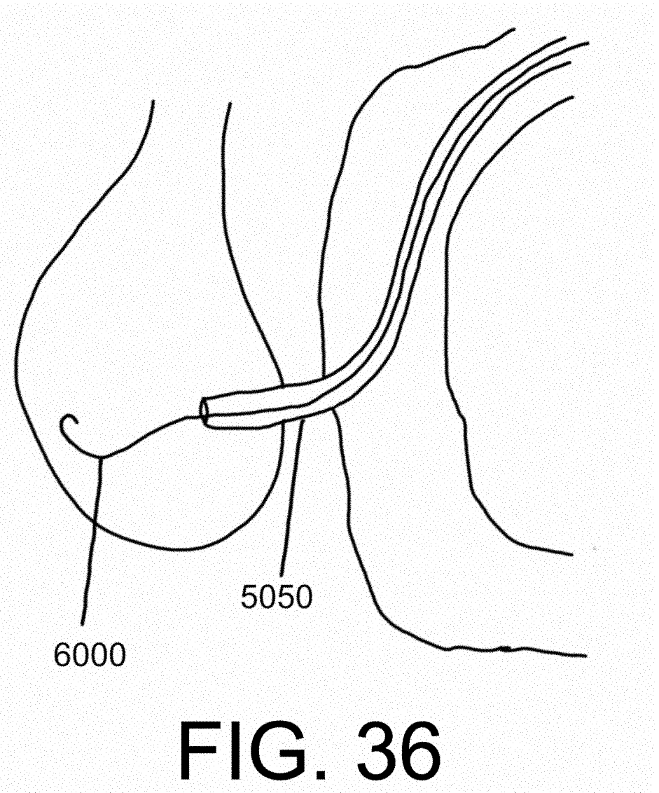

FIG. 36 depicts an endoscopic catheter 5050 that pierces through the duodenum into the gallbladder and introduces a guidewire 6000 into the lumen of the gallbladder.

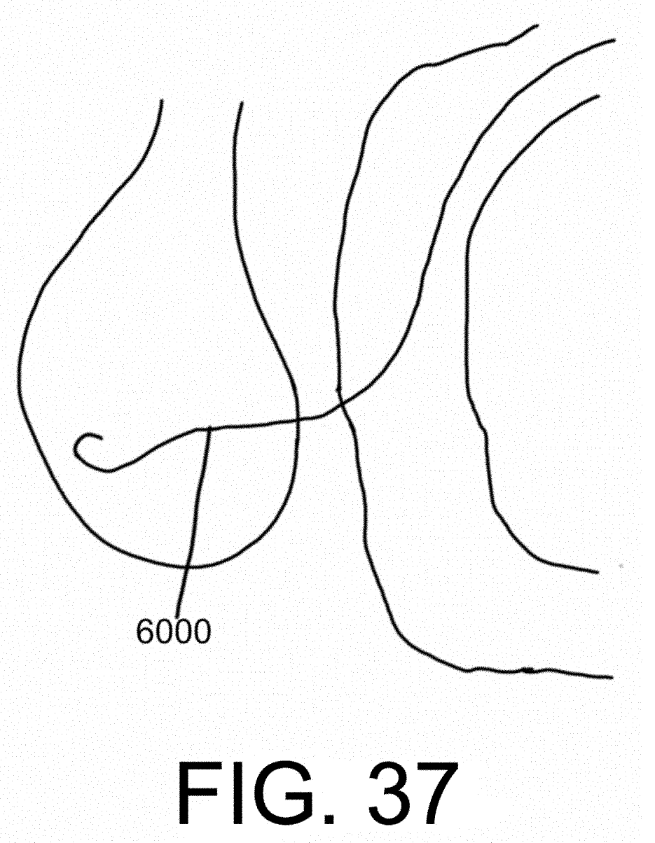

FIG. 37 depicts the guidewire 6000 left behind extending from the lumen of the duodenum into the lumen of the gallbladder after the endoscopic catheter has been removed.

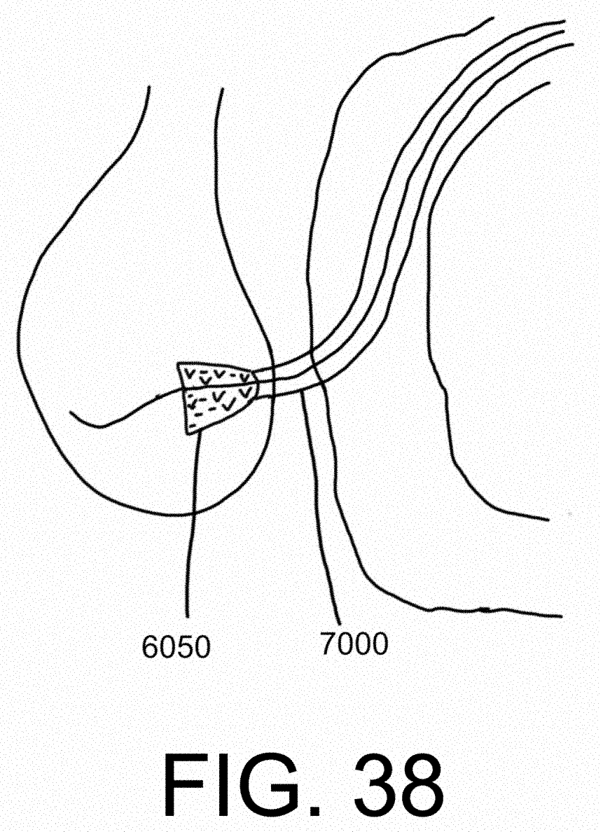

FIG. 38 depicts the unsheathing and partial deployment of an active textile endograft 6050 though a deployment catheter 7000 extending from the duodenum to the gallbladder over the previously placed guidewire.

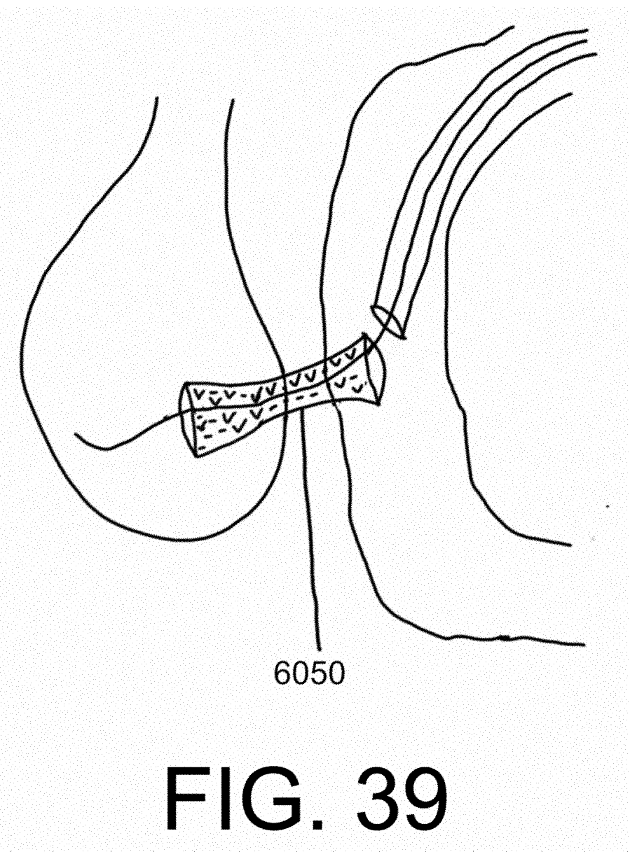

FIG. 39 depicts the complete deployment of the active textile endograft 6050 over a guidewire extending from the lumens of the duodenum and gallbladder in a martensite state. The endograft has two different active textile patterns with a specific pattern at its ends and a specific pattern in its middle portion.

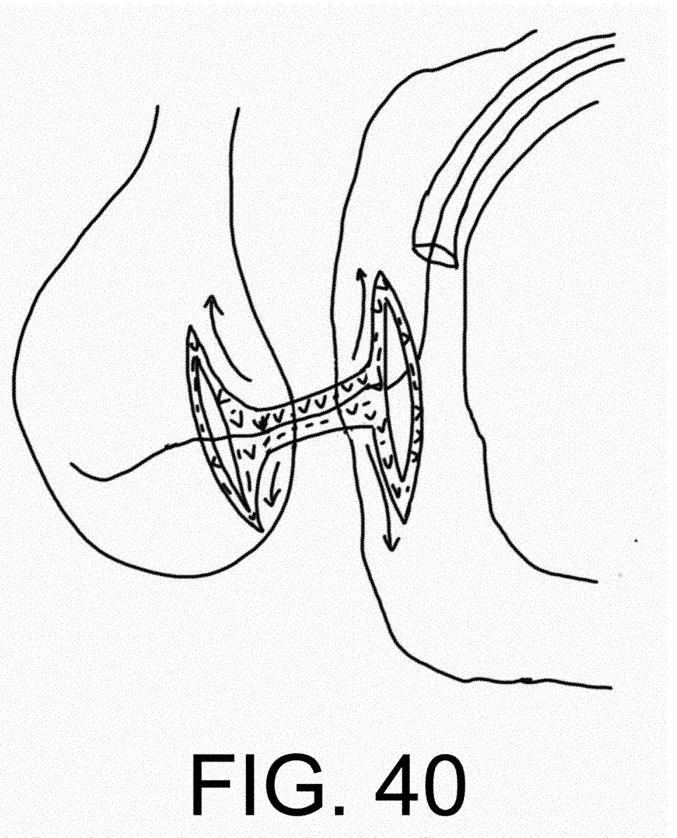

FIG. 40 depicts the activation of the ends of the endograft leading to outward flaring, flowering, flattening and near apposition of the ends with the inner walls of the gallbladder and inner wall of the duodenum. The shape changing motion is depicted by arrows in FIG. 40.

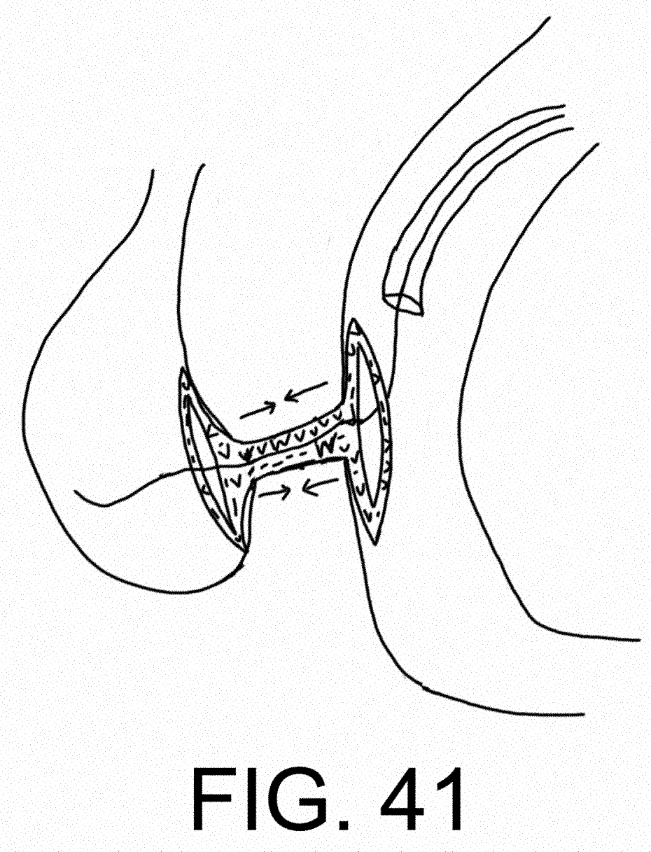

FIG. 41 depicts the activation of the middle portion of the endograft leading to foreshortening of the endograft and associated pulling together of the outer wall of the gallbladder and adjacent outer wall of the duodenum. The shape changing motion is depicted by arrows in FIG. 41.

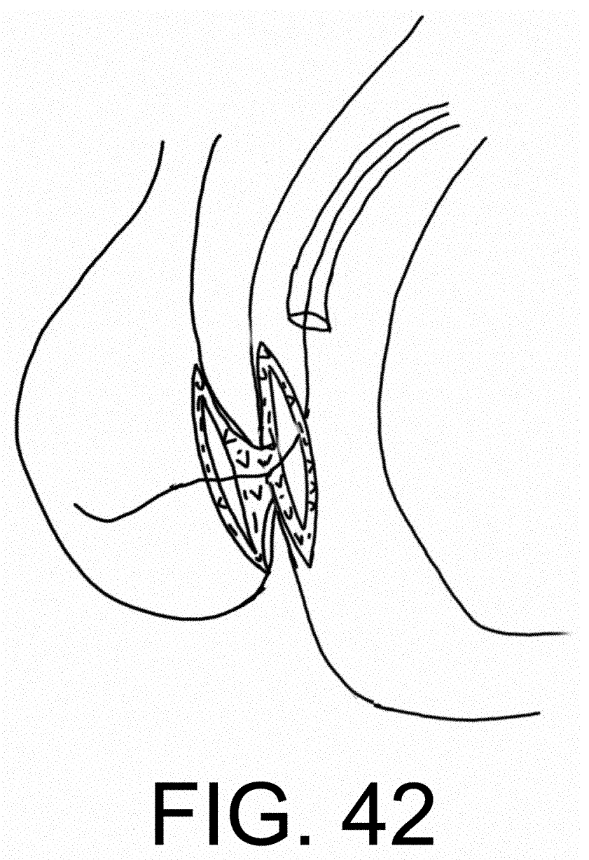

FIG. 42 shows the final shape of the active textile endograft and close position of the gallbladder and duodenum in the creation of an anastomosis between these two structures. In addition to creating an anastomosis, this embodiment can also be used to occlude existing anastomoses or existing channels for communication between hollow organs or tubular anatomical structures by eliminating the lumen of the endograft.

A fifth embodiment is an active textile sheet that is stretched and tightly rolled and then mounted and constrained on a delivery device and deployed inside the body. It is then unconstrained and activated. Upon activation the active textile sheet curls into a scroll and creates a tubular endograft. This shape changing behavior is similar to that depicted by a stockinette sheet in FIGS. 4F and 4G.

The stretching and tight rolling of the active textile sheet on the delivery device will allow for decreasing the profile of the delivery device when introduced into the body. A sheet can be stretched and tightened to higher degree than a tubular endograft because it lacks the 360 degrees of fabric and metal matrix which composes an endograft.

Therefore smaller puncture sites into the body would be required to deliver the endograft. This would decrease the risk of excessive bleeding at the puncture sites. Furthermore fenestrations, branches, and limbs could be incorporated in this embodiment of the endograft.

The fifth embodiment is depicted in the following figures:

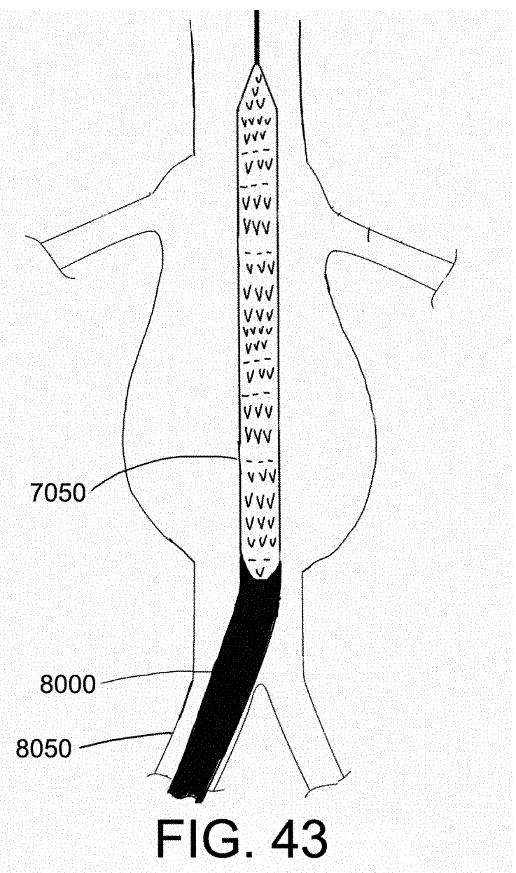

FIG. 43 depicts an active textile sheet 7050 stretched, tightly rolled and then mounted and constrained on a delivery device 8000. The active textile sheet is delivered into the abdominal aorta through the right common iliac artery 8050 via a standard puncture in the right common femoral artery.

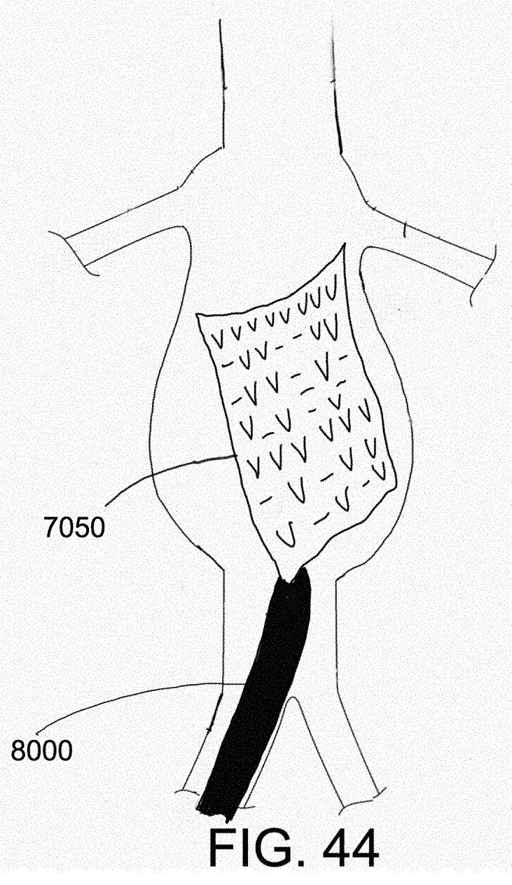

FIG. 44 depicts the removal of the constraining mechanism and unfurling of the active textile sheet in the lumen of the abdominal aorta. The sheet is shorter in length than its constrained stretched state from earlier.

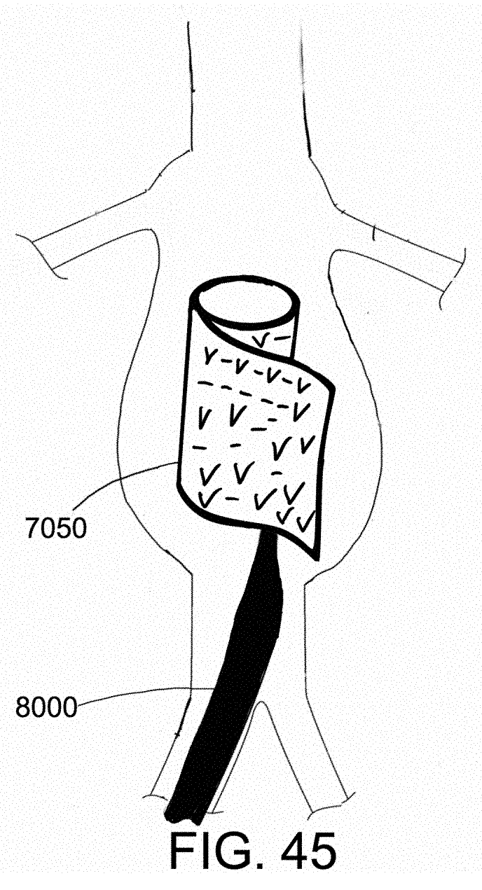

FIG. 45 depicts activation of the active textile sheet leading to a scrolling motion within the sheet and a shape change of the sheet into an incomplete tubular structure.

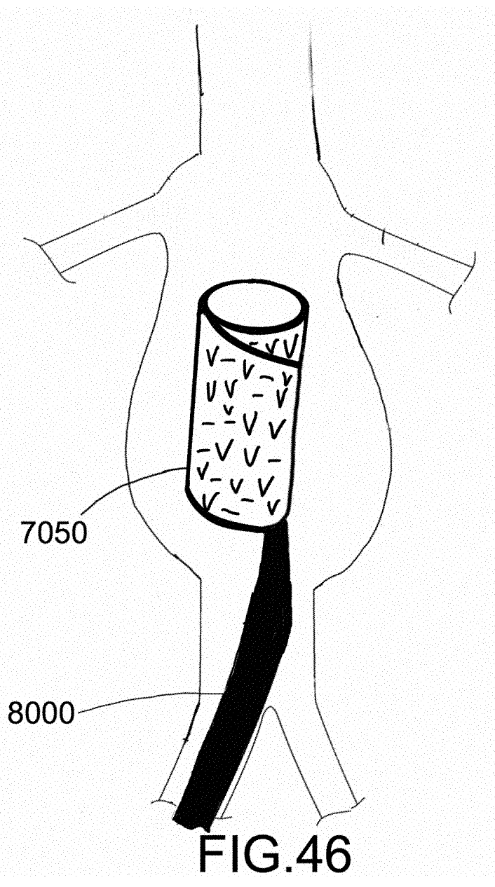

FIG. 46 depicts the final stage of activation on the active textile sheet resulting in its transformation into the complete tubular structure of an endograft.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A tubular or branched tubular endograft also known as a stentgraft or covered stent that is composed in part or in total of an active textile material configured in one or more architectural patterns that allows the endograft or parts of the endograft or components of the endograft such as fenestrations, limbs, branches, gates, trunks or other extensions to intrinsically change shape or engage in different motions following activation of the active textile material.

2. An endograft as in claim 1 in which the active textile material can be configured in one or more of the same architectural pattern or one or more different architectural patterns to achieve one type of shape change or many different types of shape changes in the endograft and in its parts and components.

3. An endograft as in claim 1 in which among the many architectural patterns the active textile material is configured into include knit patterns such as stockinette, garter, welt, vertically striped or ribbed, diagonally striped or seed, in addition to grid patterns such as stockinette and rib, in addition to restructured grid patterns such as stockinette belt, garter belt, I-cord, garter triangle and cable.

4. An endograft as in claim 1 in which the components can include one or more fenestrations, branches, limbs, gates, trunks, extensions or any other appendages associated with the endograft that alter the basic tubular shape of the endograft.

5. An endograft as in claim 1 in which the active textile material configured in one or more architectural patterns is incorporated in different parts of the endograft including components such as fenestrations, branches, limbs, trunks or other extensions that upon activation causes more than one type of shape change or motion in different parts of the endograft or in the components of the endograft.

6. An endograft as in claim 1 in which the active textile material configured in one or more architectural patterns is incorporated into a fenestration of the endograft which upon activation the active textile material causes expansion or narrowing of the fenestration.

7. An endograft as in claim 1 in which the active textile material configured in one or more architectural patterns is incorporated into a branch or limb of the endograft and upon activation, the active textile material causes expansion or narrowing and elongation or foreshortening of the branch or limb.

8. An endograft as in claim 7 in which the active textile material incorporated in the said branch or limb is configured in at least two different architectural patterns in different parts of the branch and limb that upon activation causes more than one type of shape change or motion in different parts of the branch and limb.

9. An endograft as in claim 1 in which the active textile material configured in an architectural pattern is incorporated into the ends of the endograft which upon activation causes flowering, flaring, funneling, flattening type motions at the ends leading to complete apposition of the ends of the endograft with adjacent surfaces to create a tight seal.

10. An endograft as in claim 1 in which the active textile material configured in an architectural pattern is incorporated in the middle of the endograft and causes foreshortening of the endograft and associated pulling together of the ends of the endograft.

11. An endograft as in claims 9 and 10 which contains active textile material configured in two different architectural patterns in the middle and ends of the endograft which upon activation causes flaring, funneling, flowering, flattening type motions at the ends and foreshortening of the middle of the endograft.

12. An endograft as in claim 1 composed of active textile material that is created inside the body upon activation of an active textile sheet that transforms into the said endograft.

13. A method for creating an exclusive channel for blood flow from an endograft to a branch vessel in which a balloon or a stentgraft is passed from the endograft into the lumen of the branch vessel through an active textile fenestration located on the endograft which upon activation the active textile fenestration narrows and tightens around the balloon or the stentgraft to form a tight seal around the balloon or the stentgraft creating an exclusive channel for the flow of blood from the endograft to the branch vessel and excluding flow into an aneurysm sac.

14. A method for creating an exclusive channel for blood flow from an endograft to a branch vessel in which a stentgraft is passed from the endograft into the lumen of the branch vessel through an active textile branch located on the endograft which upon activation the active textile branch narrows and tightens around the stentgraft to form a tight seal around the stentgraft creating an exclusive channel for the flow of blood from the endograft to the branch vessel and excluding flow into an aneurysm sac.

15. A method for creating an exclusive channel for blood flow from an endograft to a branch vessel in which a balloon is passed from the endograft into the lumen of the branch vessel through an active textile branch located on the endograft which upon activation the active textile branch elongates and extends from the endograft around the balloon to the ostium of the branch vessel and the end of the active textile branch closest to the ostium of the branch vessel undergoes a flowering, flaring, or flattening type shape change around the ostium to appose the tissue around the ostium to create a seal around the ostium creating an exclusive channel for the flow of blood from the endograft to the branch vessel and excluding flow into an aneurysm sac.

16. A method of connecting two components of an endograft in which a non-active textile tubular component is passed through an active textile tubular component and upon activation the active textile tubular component narrows and tightens around the non-active textile component to form a tight seal between the components.

17. A method for connecting an endograft with a fenestration to a tubular active textile endograft component in which the tubular active textile endograft component is passed through the fenestration and upon activation the end of tubular active textile endograft component extending inside the lumen of the endograft undergoes a flowering, flaring, or flattening type shape change to appose the inner wall of the endograft around the ostium of the fenestration achieving a seal with the inner wall.

18. A method for creating an anastomosis or connection between the lumens of two hollow organs or tubular anatomical structures in which an active textile endograft composed of different architectural patterns at its ends and in its middle is passed from the lumen one hollow organ or tubular anatomical structure into the lumen of the second hollow organ or tubular anatomical structure and upon activation the ends undergo a flowering, flaring, flattening type shape change to appose the inner walls of the hollow organs or tubular anatomical structures and the middle foreshortens to pull the hollow organs or tubular anatomical structures together.

19. A method as in claim 18 for creating an anastomosis or connection between the lumens of two endografts in which an active textile endograft composed of different architectural patterns at its ends and in its middle is passed from the lumen one endograft into the lumen of the second endograft and upon activation the ends of the active textile endograft undergo a flowering, flaring, flattening type shape change to appose the inner walls of the endografts and the middle foreshortens to pull the two endografts together.
